(19) United States
(12) Patent Application Publication
(10) Pub. No.: US 2006/0241084 A1
(43) Pub. Date: Oct. 26, 2006

(54) Inhibition of vascular endothelial growth factor

(75) Inventors: Chaim M. Roifman, Ontario (CA); Amos J. Simon, Yehud (IL); Peter Demin, Toronto (CA); Olga Rounova, Toronto (CA)

Correspondence Address: FISH & NEAVE IP GROUP;ROPES & GRAY LLP, ONE INTERNATIONAL PLACE, BOSTON, MA, 02110-2624, US

(73) Assignee: THE HOSPITAL FOR SICK CHILDREN, TORONTO, ON (CA)

(21) Appl. No.: 10/530,800

(22) PCT Filed: Oct. 10, 2003
(86) PCT No.: PCT/CA03/01558

§ 371 Date: Mar. 13, 2006

Related U.S. Application Data
(60) Provisional application No. 60/417,642, filed on Oct. 11, 2002.

(30) Foreign Application Priority Data
Oct. 11, 2002 (CA) 2407755

(51) Int. Cl. A61K 31/537; A61K 31/675; A61K 31/40; A61K 31/277

(52) U.S. Cl. 514/90; 514/114; 514/519; 514/521; 514/408; 514/237.5

(57) ABSTRACT

This invention relates to therapeutic organic compounds and inhibition of secretion of vascular endothelial growth factor (VEGF) and its effects, including angiogenesis.

### FIELD OF THE INVENTION

This invention relates to therapeutic organic compounds and inhibition of secretion of vascular endothelial growth factor (VEGF) and its effects, including angiogenesis.

### BACKGROUND OF THE INVENTION

VEGF is a disulfide-linked, dimeric glycoprotein with multifunctional properties. There are at least 5 isoforms derived from alternative splicing of a single gene, encoding proteins of 121, 145, 165, 189, and 206 amino acid residues. The isoforms differ in their degree of heparin-binding and sequestration in the extracellular matrix (ECM). VEGF (also known as VEGF-A) belongs to a family of growth factors that includes placental growth factor (PlGF), VEGF-B, VEGF-C, VEGF-D, and VEGF-E.

VEGF is potent stimulator of angiogenesis, the formation of new capillaries from pre-existing blood vessels. VEGF is an endothelial cell-specific mitogen and, further, VEGF induces non-proliferative endothelial cell activities involved in the angiogenic process, cell migration and invasion. VEGF induces secretion of proteolytic enzymes which degrade the basement membrane and extracellular matrix, nitric oxide release, expression of adhesion molecules, and cell morphological changes. VEGF acts as a survival factor for endothelial cells by inducing the expression of anti-apoptotic proteins.

VEGF is also a potent stimulator of vascular permeability. Discovered separately as a vascular permeability factor (VPF), VEGF causes vascular leakage with a potency 50 000 times that of histamine. Excessive vascular permeability may contribute to angiogenesis by enhancing protein extravasation, or it may represent a distinct factor in disease processes by leading to edema and swelling.

VEGF exerts its effects primarily via two endothelial receptor tyrosine kinases, VEGFR-1 (Flt-1) and VEGFR-2 (KDR/Flk-1). VEGFR-1 and -2 are thought to mediate different signal transduction functions. VEGFR-1 binds VEGF, VEGF-B, and PlGF; VEGFR-2 binds VEGF, VEGF-C, VEGF-D, and VEGF-E. VEGFR-1 shows higher affinity to VEGF. It is hypothesized that VEGFR-2, which is expressed almost exclusively in proliferating vessels, is responsible for driving endothelial cell angiogenic processes. VEGFR-1, which is expressed in hematopoietic stem cells and inflammatory cells such as monocytes and macrophages in addition to endothelial cells, is believed to mediate the activation and recruitment of these cells in response to VEGF and PlGF.

Through its angiogenic, permeability-enhancing and inflammatory effects, VEGF has been implicated in the pathophysiology of several diseases, including cancers, ocular diseases, rheumatoid arthritis, endometriosis, psoriasis, and atherosclerosis.

The critical role of tumour angiogenesis in the progression of cancer is well known. Neovascularization is necessary for the development of solid tumours beyond a few cubic millimetres in size as new blood vessels are required to provide oxygen and nutrients to the tumour cells; the new blood supply also allows tumour cells to enter the circulation and metastasize. VEGF expression is markedly upregulated in tumours and VEGF receptors are upregulated on the tumour endothelium. Numerous studies have demonstrated a correlation between tumour vascularity and metastasis or patient prognosis. In addition to solid tumours, angiogenesis has been reported to play a role in hematological disorders such as leukemias, lymphomas and myeloproliferative disorders, in which there is increased bone marrow vascularity and elevated levels of angiogenic factors. A role for VEGF in the development of ascites and pleural effusion, which cause difficult symptoms for advanced-stage cancer patients, has also been postulated.

Ischemic retinopathies, such as diabetic retinopathy and retinopathy of prematurity, are leading causes of blindness in adults and children, respectively, in the developed world. These diseases are marked by retinal neovascularization. Increased levels of VEGF are found in the vitreous and retina of patients with diabetic retinopathy. VEGF is also increased in diabetic retinal tissue without overt retinopathy and so may also play a role in the early development of the disease by mediating breakdown of the blood-retinal barrier and increased retinal vascular permeability, leading to vessel leakage and macular edema. In other eye conditions such as age-related macular degeneration, a significant cause of vision loss in aging populations, choroidal neovascularization occurs which has been shown to involve overexpression of VEGF by retinal pigment epithelial cells and choroidal fibroblasts.

In rheumatoid arthritis, an increased vascular supply to the synovium is thought to underlie the expansion of the synovial lining of joints and the development of joint destruction. Angiogenesis is involved in the formation and maintenance of the highly vascularized pannus. Increased serum VEGF levels in patients are associated with disease activity. VEGF expression in rheumatoid synovial cells such as fibroblasts or activated leukocytes is involved in the angiogenic process in rheumatoid arthritis, and may also act as a vascular permeability factor, thus increasing edema and joint swelling.

Endometriosis is characterized by significant vascularization within and surrounding ectopic endometrial tissue. A new blood supply is thought to be essential for the survival of the ectopic endometrial implant and the development of the disease. There are elevated levels of VEGF in the peritoneal fluid of women with endometriosis, secreted by peritoneal fluid and ectopic tissue macrophages.

Upregulation of VEGF and VEGF receptors occurs in psoriasis and other skin diseases. Increased circulating VEGF, seen in increased plasma VEGF levels, may cause systemic vascular hyperpermeability. Upregulated VEGF and VEGF receptors in the blister fluids and the lesional epidermis of patients with bullous diseases (bullous pemphigoid, erythema multiforme and dermatitis herptiformis) suggests that VEGF is a factor in the development hyperpermeable dermal microvessels and papillary edema.

A possible role for VEGF has been proposed in atherosclerosis. Neovascularization occurs in atherosclerotic plaques and may be required for plaque progression. Raised levels of VEGF, which are expressed by neovascular endothelial cells, smooth muscle cells and inflammatory cells, are found in patients with arterial disease. In animal models, VEGF has been shown to increase atherosclerotic plaque formation and the numbers of macrophages, while inhibition of VEGFR-1 has been described as reducing atherosclerotic plaque growth through inhibition of inflammatory cell infiltration.

Accordingly, VEGF inhibitors may provide an effective treatment for these diseases and other diseases related to VEGF.

### SUMMARY OF THE INVENTION

The present invention is based on the unexpected discovery that certain compounds, referred to herein as “VEGF inhibitors”, are capable of inhibiting the activity of VEGF. While not wishing to be bound by any particular theory, the subject VEGF inhibitors are believed to antagonize VEGF by reducing the level of secretion of the active protein.

In its broadest aspect, the invention relates to inhibition of secretion of VEGF.

In one aspect, the invention provides a method of inhibiting secretion of VEGF, in an animal in need of such inhibition, comprising administering to the animal an effective amount of a compound as disclosed herein.

In another aspect, the invention provides a method of inhibiting effects of VEGF, including angiogenesis, and a method of treating a disorder related to VEGF in an animal in need of such inhibition or treatment. In various embodiments the animal is a human patient and the disorder is cancer, rheumatoid arthritis, retinopathy and atherosclerosis.

In another aspect, the invention relates to use of compounds disclosed herein to inhibit VEGF secretion or effects of VEGF or to treat a disorder related to VEGF. The invention also relates to use of compounds disclosed herein to prepare a medicament to inhibit VEGF secretion, or effects of VEGF or to treat a disorder related to VEGF.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

FIG. 1 is a graph showing inhibition of VEGF secretion from HTB-133 (KDR+) and HTB-131 (KDR−) breast cancer cell lines by CR-4.

FIG. 2 is a graph showing inhibition of VEGF secretion from breast cancer MDA-231 cell line by CR4.

FIG. 3 is a graph showing inhibition of VEGF secretion from HTB-72 melanoma cell line by CR4.

FIG. 4 is a graph showing inhibition of VEGF secretion from CR2-1730 HUVEC cell line by CR4.

FIG. 5 is a graph showing inhibition of HUVEC growth by CR-4 induced VEGF depleted media of HTB-133 (KDR+) and HTB-131 (KDR−) breast cancer cell lines and rescue by recombinant human VEGF (10 ng/ml).

FIG. 6 is a graph showing inhibition of VEGF secretion from breast cancer cell lines HTB-133 and MDA-231 by CR11.

FIG. 7 is a graph showing inhibition of VEGF secretion from breast cancer cell lines HTB-133 and MDA-231 by CR19.

### DETAILED DESCRIPTION OF THE INVENTION

The inventors have discovered that certain compounds inhibit VEGF activity. These compounds inhibit, in a dose dependent manner, VEGF secretion from breast cancer cell lines, melanoma cell line and human umbilical vascular endothelial cell line. These compounds therefore provide a new therapeutic approach that target expression and secretion of VEGF.

The compounds, by inhibiting VEGF secretion, also inhibit the effects of VEGF. VEGF, first identified as an endothelial cell specific growth factor, stimulates endothelial cell proliferation. CR4, by inhibiting secretion of VEGF, was found to inhibit endothelial cell proliferation. In particular, when CR4 is added to breast cancer cell lines which normally secrete VEGF, the secretion of VEGF to the medium is inhibited and the medium is unable to stimulate proliferation of human umbilical vascular endothelial primary cells (these cells do not secrete detectable amount of VEGF). The inhibition of endothelial cell growth is however rescued if recombinant VEGF is added to the medium, confirming that inhibition results from inhibition of VEGF secretion. The inhibition of cell growth is also CR4 dose dependent, with an IC50 value of about 20-30 nM. The effective dose is therefore similar to that required for inhibition of VEGF secretion and further confirms that inhibition of cell growth results from inhibition of VEGF secretion.

These results indicate that compounds disclosed herein therefore can be used to inhibit the effects of VEGF, including both paracrine and autocrine activities. Furthermore, while VEGF is an essential part of normal embryonic development, repair, tissue regeneration, female reproductive cycle and other physiological processes, the effects of VEGF is associated with variety of disorders. In many of these disorders, VEGF expression/levels are upregulated. The compounds therefore may also be used to treat these disorders. By way of an example, VEGF plays a central role in angiogenesis (and vasculogenesis during embryonic development), the formation of new blood vessels, and it is known that angiogenesis is critical to growth and metastasis of tumour cells in the progression of cancer. The compounds therefore may be used to treat cancer, including, by inhibiting tumour growth.

The invention therefore provides a method of inhibiting VEGF secretion comprising administering to an animal in need of such inhibition an effective amount of a compound as disclosed herein. The invention also provides a method of inhibiting effects of VEGF in an animal in need of such inhibition. The term “effects of VEGF” is used to refer to effects resulting from, or mediated by VEGF activity, including via its receptor(s), and include angiogenesis, vasculogenesis, arteriogenesis, vascular permeability, and inflammation. The term inhibition or inhibiting or like terms are used to broadly refer to any reduction of targeted characteristic which is statistically significant when compared to a control (i.e., no administration of the compounds) as can be measured using techniques known in the art.

The animal may be a human patient suffering from disorders related to VEGF. It is meant by such disorders any disorder in which VEGF is believed to play a role in the progression or symptoms of the disorder or in which VEGF expression and/or levels are upregulated. Cancers having VEGF-related components to the disease include: solid tumors cancers such as breast, pancreatic, colon and brain cancer, melanoma; hemangioma; Karposi's sarcoma; and hematological malignancy, including leukemia, lymphoproliferative disorders and myeloproliferative disorders.

The present invention may be used to treat animals and patients with aberrant angiogenesis, such as that contributing to a variety of diseases and disorders. The most prevalent and/or clinically important of these, outside the field of cancer treatment, include arthritis (such as rheumatoid arthritis), psoriasis, atherosclerosis, disorders involving unwanted ocular vascularization, Grave's disease, vascular restenosis, including restenosis following angioplasty, arteriovenous malformations (AVM), renal vein occlusion, and neovascular glaucoma. Other potential targets for intervention include angiofibroma, atherosclerotic plaques, corneal graft neovascularization, hemophilic joints, hypertrophic scars, osler-weber syndrome, pyogenic granuloma retrolental fibroplasia, scleroderma, trachoma, vascular adhesions, synovitis, dermatitis, various other inflammatory diseases and disorders, and even endometriosis. Further diseases and disorders that are treatable by the invention, and the unifying basis of such angiogenic disorders, are set forth below.

One disease in which angiogenesis is involved is rheumatoid arthritis, wherein the blood vessels in the synovial lining of the joints undergo angiogenesis. In addition to forming new vascular networks, the endothelial cells release factors and reactive oxygen species that lead to pannus growth and cartilage destruction. The factors involved in angiogenesis, such as VEGF, are understood to contribute to, and help maintain, the chronically inflamed state of rheumatoid arthritis. For instance, VEGF is constitutively expressed in the synovium of rheumatoid arthritis. The known anti-rheumatic drug, bucillamine (BUC), was shown to include within its mechanism of action the inhibition of VEGF production by synovial cells. VEGF and other factors associated with angiogenesis also have a role in osteoarthritis, contributing to the destruction of the joint. The present invention specifically contemplates the use of the subject VEGF inhibitors as part of an anti-arthritic therapy.

Many diseases with an angiogenic component are associated with the eye and for which VEGF activity has been implicated in the pathogenesis of disease. In certain embodiments, the subject VEGF inhibitors can be used in the treatment of ocular neovascular diseases. By “ocular neovascular disease” is meant a disease characterized by ocular neovascularization, i.e. the development of abnormal blood vessels in the eye of a patient, and include corneal neovascularization.

To further illustrate, diseases associated with ocular neovascularization that can be treated using the subject VEGF inhibitors include, but are not limited to, corneal neovascularization, retinal neovascularization, choroidal neovascularization, and include such disorders as retinopathy (such as diabetic, ischemic and/or retinopathy of prematurity), macular degeneration (such as age-related), corneal graft rejection, neovascular glaucoma, retrolental fibroplasia, epidemic keratoconjunctivitis, Vitamin A deficiency, contact lens overwear, atopic keratitis, superior limbic keratitis, pterygium keratitis sicca, sjogrens, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi sarcoma, Mooren ulcer, Terrien's marginal degeneration, marginal keratolysis, trauma, rheumatoid arthritis, systemic lupus, polyarteritis, Wegeners sarcoidosis, Scleritis, Steven's Johnson disease, periphigoid radial keratotomy, diabetic macular edema, diabetic retina ischemia, and diabetic retinal edema.

In certain preferred embodiments, the subject VEGF inhibitors are used as part of a treatment for diseases associated with retinal/choroidal neovascularization, such as diabetic retinopathy, macular degeneration, sickle cell anemia, sarcoid, syphilis, pseudoxanthoma elasticum, Pagets disease, vein occlusion, artery occlusion, carotid obstructive disease, chronic uveitis/vitritis, mycobacterial infections, Lyme's disease, systemic lupus erythematosis, retinopathy of prematurity, Eales disease, Bechets disease, infections causing a retinitis or choroiditis, presumed ocular histoplasmosis, Bests disease, myopia, optic pits, Stargarts disease, pars planitis, chronic retinal detachment, hyperviscosity syndromes, toxoplasmosis, trauma and post-laser complications.

Other ocular disorders and diseases that can be treated according to the present invention include, but are not limited to, diseases associated with rubeosis or other iris neovascularization, and diseases caused by the abnormal proliferation of fibrovascular or fibrous tissue including all forms of proliferative vitreoretinopathy, whether or not associated with diabetes.

Other diseases include, but are not limited to, diseases associated with rubeosis (neovascularization of the angle) and diseases caused by the abnormal proliferation of fibrovascular or fibrous tissue including all forms of proliferative vitreoretinopathy.

The subject VEGF inhibitors can be used alone or in combination with other therapeutic regimens directed to treating ocular diseases. For instance, the present invention provides a method of treating an ocular neovascular disease which involves administering to a patient a VEGF inhibitor and treating the patient with phototherapy (e.g., PDT) or with other therapies, such as photocoagulation, that destroy abnormal blood vessels in the eye.

Chronic inflammation also involves pathological angiogenesis and can be treated with a program that includes the subject VEGF inhibitors. Such disease states as ulcerative colitis and Crohn's disease show histological changes with the ingrowth of new blood vessels into the inflamed tissues.

Bartonellosis, a bacterial infection found in South America, can result in a chronic stage that is characterized by proliferation of vascular endothelial cells, and may be amenable to treatment including the subject VEGF inhibitors.

Another pathological role associated with angiogenesis is found in atherosclerosis. The plaques formed within the lumen of blood vessels have been shown to have angiogenic stipulatory activity. VEGF expression in human coronary atherosclerotic lesions has been demonstrated. This evidences the pathophysiological significance of VEGF in the progression of human coronary atherosclerosis, as well as in recanalization processes in obstructive coronary diseases. The subject VEGF inhibitors can be used as part of an-effective treatment for such conditions.

One of the most frequent angiogenic diseases of childhood is the hemangioma. In most cases, the tumors are benign and regress without intervention. In more severe cases, the tumors progress to large cavernous and infiltrative forms and create clinical complications. Systemic forms of hemangiomas, the hemangiomatoses, have a high mortality rate. Therapy-resistant hemangiomas exist that cannot be treated with therapeutics currently in use. The subject VEGF inhibitors can be used in the treatment of hemangiomas.

Angiogenesis is also responsible for damage found in hereditary diseases such as Osler-Weber-Rendu disease, or hereditary hemorrhagic telangiectasia. This is an inherited disease characterized by multiple small angiomas, tumors of blood or. lymph vessels. The angiomas are found in the skin and mucous membranes, often accompanied by epistaxis (nosebleeds) or gastrointestinal bleeding and sometimes with pulmonary or hepatic arteriovenous fistula. The subject VEGF inhibitors can be used in the treatment of angiomas.

Angiogenesis is also involved in normal physiological processes such as reproduction and wound healing. Angiogenesis is an important step in ovulation and also in implantation of the blastula after fertilization. Prevention of angiogenesis with the subject VEGF inhibitors can be used to induce amenorrhea, to block ovulation or to prevent implantation by the blastula.

In wound healing, excessive repair or fibroplasia can be a detrimental side effect of surgical procedures and may be caused or exacerbated by angiogenesis. Adhesions are a frequent complication of surgery and lead to problems such as small bowel obstruction. The subject VEGF inhibitors can be used post-operatively to influence wound healing processes.

Diseases and disorders characterized by undesirable vascular permeability can also be treated by the present invention. These include edema associated with brain tumors, ascites associated with malignancies, Meigs' syndrome, lung inflammation, nepbrotic syndrome, pericardial effusion and pleural effusion, as disclosed in WO 98/16551.

Thus, the invention provides a method of treating a disorder related to VEGF comprising administering an effective amount of a compound as disclosed herein to an animal in need of such treatment.

Compounds useful in the compositions and methods disclosed herein include compounds of Formula I, and salts, solvates or hydrates thereof: 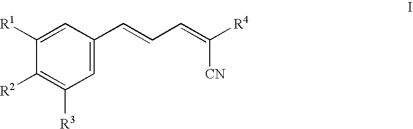
wherein
R¹ and R² are each independently selected from H, OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, O—Si(C₁₋₆alkyl)(C₁₋₆alkyl)(C₁₋₆alkyl), NO₂, CF₃, OCF₃ and halo;
R³ is selected from H, OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, O—Si(C₁₋₆alkyl)(C₁₋₆alkyl)(C₁₋₆alkyl), NO₂, halo and CH₂—S—(CH₂)ₙ Ar;
R⁴ is selected from C(X)R⁵, SO₃Ar, NH₂, NH—C₁₋₆alkyl, N(C₁₋₆alkyl)(C₁₋₆alkyl), P(O)(OH)₂, P(O)(OC₁₋₆alkyl)₂, and C(NH₂)═C(CN)₂;
X is selected from O, S, NH and N—C₁₋₆alkyl;
R⁵ is selected from NH₂, OH, NH(CH₂)ₚAr, NH(CH₂)ₚOH, (CH₂)ₚOC₁₋₆alkyl, C₁₋₆alkyl, C₁₋₆alkoxy, NHNH₂, NHC(O)NH₂, NHC(O)C₁₋₆alkoxy, N-morpholino and N-pyrrolidino; and
Ar is an aromatic or heteroaromatic group, unsubstituted or substituted with 1-4 substituents independently selected from OH, C₁₋₆alkyl, C₁₋₆alkoxy, NH₂, NH—C₁₋₆alkyl, N(C₁₋₆alkyl)(C₁₋₆allyl), SH, S—C₁₋₆alkyl, NO₂, CF₃, OCF₃ and halo;
n is 0 to 4;
m is 1 to 4; and
p is 1-4.

In embodiments of the invention, compounds of Formula I are those in which R¹ and R² are each independently selected from H, OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, O—Si(C₁₋₆alkyl)(C₁₋₆alkyl)(C₁₋₆alkyl), NO₂, CF₃, OCF₃ and halo. In preferred embodiments, R¹ and R² are each independently selected from H, OH, C₁₋₄alkyl, C₁₋₄alkoxy, NH₂, NH—C₁₋₄alkyl, SH, S—C₁₋₄alkyl, O—Si(C₁₋₄alkyl)(C₁₋₄alkyl)(C₁₋₄alkyl), NO₂, CF₃, OCF₃ and halo. In more preferred embodiments, R¹ and R² are each independently selected from H, OH, OCH₃, O—Si(CH₃)₂(^{t}Bu), S-Me, SH, and NO₂. In the most preferred embodiment of the present invention R¹ and R² are both OH or OCH₃ or R¹ is OCH₃ and R² is OH.

In further embodiments of the present invention, the compounds of Formula I include those in which R³ is selected from H, OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, O—Si(C₁₋₆alkyl)(C₁₋₆alkyl)(C₁₋₆alkyl), NO₂, halo and CH₂—S—(CH₂)ₙAr (where n is 0-4). In preferred embodiments, R³ is selected from H, OH, C₁₋₄alkyl, C₁₋₄alkoxy, NH₂, NH—C₁₋₄alkyl, N(C₁₋₄alkyl)(C₁₋₄alkyl), SH, S—C₁₋₄alkyl, NO₂ and halo. In a more preferred embodiment, R³ is selected from H, OH, OCH₃, SH, SMe, NO₂ and halo. In the most preferred embodiment, R³ is selected from H, OH and OCH₃.

Exemplary compounds include compounds of Formula I wherein R⁴ is selected from C(X)R⁵, SO₃Ar, NH₂, NH—C₁₋₆alkyl, N(C₁₋₆alkyl)(C₁₋₆alkyl), P(O)(OH)₂, P(O)(OC₁₋₆alkyl)₂, and C(NH₂)═C(CN)₂ (where m is 1-4). In preferred embodiments, R⁴ is selected from C(X)R⁵ and C(NH₂)═C(CN)₂. More preferably, R⁴ is C(X)R⁵. When R⁴ is C(X)R⁵, embodiments of the invention include compounds where X is selected from O,S, NH and N—C₁₋₆alkyl and R⁵ is selected from NH₂, OH, NH(CH₂)ₚAr, NH(CH₂)ₚOH, (CH₂)ₚOC₁₋₆alkyl, C₁₋₆alkyl, C₁₋₆alkoxy, NHNH₂, NHC(O)NH₂, NHC(O)C₁₋₆alkoxy, N-morpholino and N-pyrrolidino (where p is 1-4). In preferred embodiments, X is O or S and R⁵ is selected from NH₂, OH, NH(CH₂)ₚAr, (CH₂)ₚOH and C₁₋₄alkoxy, (where p is 1-3). Most preferred, are compounds of Formula I wherein X is O and R⁵ is selected from NH₂, OH, NH(CH₂)ₚAr, NH(CH₂)ₚOH and OCH₃, (where p is 1-2).

Suitable compounds include compounds of Formula I wherein the term “Ar” means an unsubstituted or substituted aryl and/or heteroaryl group which, in the case of heteroaryl, may contain up to two heteroatoms, wherein the optional substituents are independently selected from OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, NO₂, CF₃, OCF₃ and halo, and includes unsubstituted or substituted phenyl, furyl, thienyl, indolyl, naphthyl, quinolyl and the like. In embodiments of the present invention, Ar is an unsubstituted phenyl group or a phenyl group substituted with 1-4 substituents optionally selected from OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, NO₂, CF₃, OCF₃ and halo. In preferred embodiments, Ar is an unsubstituted phenyl group or phenyl group substituted with 1-2 substituents optionally selected from OH, C₁₋₄alkyl, C₁₋₄alkoxy, NH₂, NH—C₁₋₄alkyl, N(C₁₋₄alkyl)(C₁₋₄alkyl), SH, S—C₁₋₄alkyl, NO₂, CF₃, OCF₃ and halo. In more preferred embodiments, Ar is an unsubstituted phenyl group or phenyl group substituted with 1-2 substituents optionally selected from OH, OCH₃, NH₂, NHCH₃, N(CH₃)₂, SH, SCH₃, CF₃, OCF₃ and halo. In the most preferred embodiment, Ar is selected from phenyl and 3,4-dihydroxyphenyl.

Other compounds useful in the compositions and methods disclosed herein include compounds of Formula II and salts, solvates and hydrates thereof: 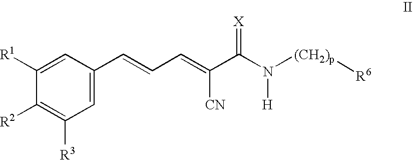
wherein
R¹ and R² are each independently selected from H, OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, O-Si(C₁₋₆alkyl)(C₁₋₆alkyl)(C₁₋₆alkyl), NO₂, CF₃, OCF₃ and halo;
R³ is selected from H, OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, O—Si(C₁₋₆alkyl)(C₁₋₆alkyl)(C₁₋₆alkyl), NO₂, halo and CH₂—S—(CH₂)ₙAr;
Ar is an aromatic or heteroaromatic group, unsubstituted or substituted with 1-4 substituents, independently selected from OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, NO₂, CF₃, OCF₃ and halo;
R⁶ is selected from Ar, OH and OC₁₋₆alkyl;
X is selected from O and S;
n is 0-4; and
p is 1-4.

In embodiments of the invention, compounds of Formula II are those in which R¹ and R² are each independently selected from H, OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, O—Si(C₁₋₆alkyl)(C₁₋₆alkyl)(C₁₋₆alkyl), NO₂, CF₃, OCF₃ and halo. In preferred embodiments, R¹ and R² are each independently selected from H, OH, C₁₋₄alkyl, C₁₋₄alkoxy, NH₂, NH—C₁₋₄alkyl, SH, S—C₁₋₄alkyl, O—Si(C₁₋₄alkyl)(C₁₋₄alkyl)(C₁₋₄alkyl), NO₂, CF₃, OCF₃ and halo. In more preferred embodiments, R¹ and R² are each independently selected from H, OH, OCH₃, O—Si(CH₃)₂(^{t}Bu), S-Me, SH, and NO₂. In the most preferred embodiment of the present invention R¹ and R² are both OH or OCH₃ or R¹ is OCH₃ and R² is OH.

In further embodiments of the present invention, the compounds of Formula II include those in which R³ is selected from H, OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, O—Si(C₁₋₆alkyl)(C₁₋₆alkyl)(C₁₋₆alkyl), NO₂, halo and CH₂—S—(CH₂)ₙAr (where n is 0-4). In preferred embodiments, R³ is selected from H, OH, C₁₋₄alkyl, C₁₋₄alkoxy, NH₂, NH—C₁₋₄alkyl, N(C₁₋₄alkyl)(C₁₋₄alkyl), SH, S—C₁₋₄alkyl, NO₂ and halo. In a more preferred embodiment, R³ is selected from H, OH, OCH₃, SH, SMe, NO₂, and halo. In the most preferred embodiment, R³ is selected from H, OH and OCH₃.

Suitable compounds include compounds of Formula II wherein the term “Ar” means an unsubstituted or substituted aryl and heteroaryl group which, in the case of heteroaryl, may contain up to two heteroatoms, wherein the optional substituents are independently selected from OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, NO₂, CF₃, OCF₃ and halo, and includes unsubstituted or substituted phenyl, furyl, thienyl, indolyl, naphthyl, quinolyl and the like. In embodiments of the present invention, Ar is an unsubstituted phenyl group or a phenyl group substituted with 1-4 substituents optionally selected from OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, NO₂, CF₃, OCF₃ and halo. In preferred embodiments, Ar is an unsubstituted phenyl group or phenyl group substituted with 1-2 substituents optionally selected from OH, C₁₋₄alkyl, C₁₋₄alkoxy, NH₂, NH—C₁₋₄alkyl, N(C₁₋₄alkyl)(C₁₋₄alkyl), SH, S—C₁₋₄alkyl, NO₂, CF₃, OCF₃ and halo. In more preferred embodiments, Ar is an unsubstituted phenyl group or phenyl group substituted with 1-2 substituents optionally selected from OH, OCH₃, NH₂, NHCH₃, N(CH₃)₂, SH, SCH₃, CF₃, OCF₃ and halo. In the most preferred embodiment, Ar is selected from phenyl and 3,4-dihydroxyphenyl.

The compounds of Formula II include those in which R⁶ is selected from Ar, OH and OC₁₋₆alkyl and p is 1-4. In preferred embodiments, R⁶ is selected from Ar and OH and p is 1-2. Most preferably, when R⁶ is Ar, p is 1 and when R⁶ is OH, p is 2. Where R⁶ is Ar, Ar means an unsubstituted or substituted aryl and/or heteroaryl group which, in the case of heteroaryl, may contain up to two heteroatoms, wherein the optional substituents are independently selected from OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, NO₂, CF₃, OCF₃ and halo, and includes unsubstituted or substituted phenyl, furyl, thienyl, indolyl, naphthyl, quinolyl and the like. In embodiments of the present invention, Ar is an unsubstituted phenyl group or a phenyl group substituted with 1-4 substituents optionally selected from OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, NO₂, CF₃, OCF₃ and halo. In preferred embodiments, Ar is an unsubstituted phenyl group or phenyl group substituted with 1-2 substituents optionally selected from OH, C₁₋₄alkyl, C₁₋₄alkoxy, NH₂, NH—C₁₋₄alkyl, N(C₁₋₄alkyl)(C₁₋₄alkyl), SH, S—C₁₋₄alkyl, NO₂, CF₃, OCF₃ and halo. In more preferred embodiments, Ar is an unsubstituted phenyl group or phenyl group substituted with 1-2 substituents optionally selected from OH, OCH₃, NH₂, NHCH₃, N(CH₃)₂, SH, SCH₃, CF₃, OCF₃ and halo. In the most preferred embodiment, Ar is selected from phenyl and 3,4-dihydroxyphenyl.

Compounds of Formula II further include those in which X is selected from O and S. In preferred embodiments, X is O.

Compounds useful in the compositions and methods disclosed herein include compounds of Formula III and salts, solvates and hydrates thereof: 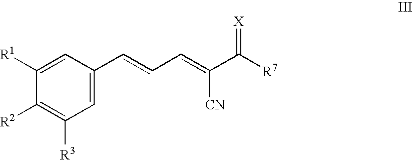
wherein
R¹ and R² are each independently selected from H, OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, O—Si(C₁₋₆alkyl)(C₁₋₆alkyl)(C₁₋₆alkyl), NO₂, CF₃, OCF₃ and halo;
R³ is selected from H, OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, O—Si(C₁₋₆alkyl)(C₁₋₆alkyl)(C₁₋₆alkyl), NO₂, halo and CH₂—S—(CH₂)ₙAr,
Ar is an aromatic or heteroaromatic group, unsubstituted or substituted with 1-4 substituents, independently selected from OH,
C₁₋₆alkyl, C₁₋₆alkoxy, NH₂, NH—C₁₋₆alkyl, N(C₁₋₆alkyl)(C₁₋₆alkyl), SH, S—C₁₋₆alkyl, NO₂, CF₃, OCF₃ and halo;
R⁷ is selected from OH, NH₂ and OC₁₋₆alkyl;
X is selected from O and S; and
n is 0-4.

In embodiments of the invention, compounds of Formula III are those in which R¹ and R² are each independently selected from H, OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, O—Si(C₁₋₆alkyl)(C₁₋₆alkyl)(C₁₋₆alkyl), NO₂, CF₃, OCF₃ and halo. In preferred embodiments, R¹ and R² are each independently selected from H, OH, C₁₋₄alkyl, C₁₋₄alkoxy, NH₂, NH—C₁₋₄alkyl, SH, S—C₁₋₄alkyl, O—Si(C₁₋₄alkyl)(C₁₋₄alkyl)(C₁₋₄alkyl), NO₂, CF₃, OCF₃ an halo. In more preferred embodiments, R¹ and R² are each independently selected from H, OH, OCH₃, O—Si(CH₃)₂(^{t}Bu), S-Me, SH, and NO₂. In the most preferred embodiment of the present invention, R¹ and R² are both OH or OCH₃ or R¹ is OCH₃ and R² is OH.

In further embodiments of the present invention, the compounds of Formula III include those in which R³ is selected from H, OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, O—Si(C₁₋₆alkyl)(C₁₋₆alkyl)(C₁₋₆alkyl), NO₂, halo and CH₂—S—(CH₂)ₙAr (where n is 0-4). In preferred embodiments, R³ is selected from H, OH, C₁₋₄alkyl, C₁₋₄alkoxy, NH₂, NH—C₁₋₄alkyl, N(C₁₋₄alkyl)(C₁₋₄alkyl), SH, S—C₁₋₄alkyl, NO₂ and halo. In a more preferred embodiment, R³ is selected from H, OH, OCH₃, SH, SMe, NO₂, and halo. In the most preferred embodiment, R³ is selected from H, OH and OCH₃.

The present invention further contemplates compounds of Formula III wherein the term “Ar” means an unsubstituted or substituted aryl and/or heteroaryl group which, in the case of heteroaryl, may contain up to two heteroatoms, wherein the optional substituents are independently selected from OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, NO₂, CF₃, OCF₃ and halo, and includes unsubstituted or substituted phenyl, furyl, thienyl, indolyl, naphthyl, quinolyl and the like. In embodiments of the present invention, Ar is an unsubstituted phenyl group or a phenyl group substituted with 1-4 substituents optionally selected from OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, NO₂, CF₃, OCF₃ and halo. In preferred embodiments, Ar is an unsubstituted phenyl group or phenyl group substituted with 1-2 substituents optionally selected from OH, C₁₋₄alkyl, C₁₋₄alkoxy, NH₂, NH—C₁₋₄alkyl, N(C₁₋₄alkyl)(C₁₋₄alkyl), SH, S—C₁₋₄alkyl, NO₂, CF₃, OCF₃ and halo. In more preferred embodiments, Ar is an unsubstituted phenyl group or phenyl group substituted with 1-2 substituents optionally selected from OH, OCH₃, NH₂, NHCH₃, N(CH₃)₂, SH, SCH₃, CF₃, OCF₃ and halo. In the most preferred embodiment, Ar is selected from phenyl and 3,4-dihydroxyphenyl.

Compounds of Formula III further include those in which R⁷ is selected from OH, NH₂ and OC₁₋₆alkyl. In preferred embodiments, R⁷ is selected from OH and NH₂.

Compounds of Formula m further include those in which X is selected from O and S. In preferred embodiments, X is O.

In specific embodiments of the present invention, suitable compounds include:
(E,E)-2-(benzylamido)-3-styrylacrylonitrile (CR1);
(E,E)-2-(benzylamido)-3-(3,4-dimethoxystyryl)acrylonitrile (CR2);
(E,E)-2-(benzylamido)-3-(3,5-dimethoxy-4-hydroxystyryl)acrylonitrile (CR3);
(E,E)-2-(benzylamido)-3-(3,4-dihydroxystyryl)acrylonitrile (CR4);
(E,E)-2-(phenylethylamido)-3-(3,4-dimethoxystyryl)acrylonitrile (CR5);
(E,E)-2-(phenylethylamido)-3-(3,5-dimethoxy-4-hydroxystyryl)acrylonitrile (CR8);
(E,E)-2-(phenylpropylamido)-3-(3,5-dimethoxy-4-hydroxystyryl)acrylonitrile (CR9);
(E,E)-2-(3,4-dihydroxybenzylamido)-3-(3,5-dimethoxy-4-hydroxystyryl)acrylonitrile (CR11);
(E,E)-2-thioacetamido-3-(3,5-dimethoxy-4-hydroxystyryl)acrylonitrile (CR12);
(E,E)-2-acetamido-3-(3,5-dimethoxy-4-hydroxystyryl)acrylonitrile (CR13);
(E,E)-2-carboxy-3-(3,5-dimethoxy-4-hydroxystyryl)acrylonitrile (CR14);
(E,E)-2-carbomethoxy-3-(3,5-dimethoxy-4-hydroxystyryl)acrylonitrile (CR15);
(E,E)-2-acetamido-3-[3,4-bis(t-butyldimethylsilyloxystyryl)]acrylonitrile (CR16);
(E,E)-2-acetamido-3-(3,4-dihydroxystyryl)acrylonitrile (CR17);
(E,E)-2-(benzylamido)-3-(3,4-bis(t-butyldimethylsilyloxystyryl)]acrylonitrile (CR18);
(E,E)-2-(3,4-dihydroxybenzylamido)-3-styrylacrylonitrile (CR19);
(E,E)-2-(3,4-dihydroxybenzylamido)-3-[3,4-bis(t-butyldimethylsilyloxystyryl)]acrylonitrile (CR20);
(E,E)-2-(3,4-dihydroxybenzylamido)-3-(3,4-dihydroxystyryl)acrylonitrile (CR21);
(E,E)-2-(β-ethanolamido)-3-(3,5-dimethoxy-4-hydroxystyryl)acrylonitrile (CR24);
(E,E)-2-(benzylamido)-3-(4-nitrostyryl)acrylonitrile (CR27);
(E,E)-2-(3,4-dihydroxybenzylamido)-3-(4-nitrostyryl)acrylonitrile (CR28); and
(E,E)-2-(1-amino-2,2-dicyanoethenyl)-3-(4-nitrostyryl)acrylonitrile (CR29).

Additional compounds useful in the compositions and methods disclosed herein include compounds of Formula IV, and salts, solvates or hydrates thereof. 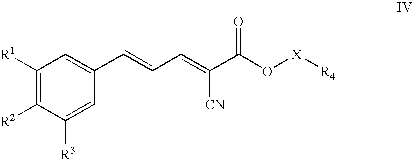
wherein
R¹, R² and R³ are each independently selected from H, OH, C₁₋₆alkyl, C₁₋₆alkoxy, NH₂, NH—C₁₋₆alkyl, N(C₁₋₆alkyl)(C₁₋₆alkyl), SH,
S—C₁₋₆alkyl, NO₂, CF₃, OCF₃ and halo;
R⁴ is unsubstituted Ar, or Ar substituted with 1-4 substituents, independently selected from C₁₋₆alkyl, C₁₋₆alkoxy and halo;
X is selected from (CH₂CH₂O)ₙ and (CH₂)ₙ, and
n=1-3.

In embodiments of the invention, compounds of Formula IV are those in which R¹, R² and R³ are each independently selected from H, OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, NO₂, CF₃, OCF₃ and halo. In preferred embodiments, R¹, R² and R³ are each independently selected from H, OH, C₁₋₄alkyl, C₁₋₄alkoxy, NH₂, NH—C₁₋₄alkyl, N(C₁₋₄alkyl)(C₁₋₄alkyl), NO₂, CF₃, OCF₃ and halo. In more preferred embodiments, R¹, R² and R³ are each independently selected from H, OH, OCH₃, NH₂, N(CH₃)₂, and NO₂. In the most preferred embodiments, R¹, R² and R³ are each independently selected from H, OH and OCH₃.

Further embodiments of the invention relate to compounds of Formula IV wherein R⁴ is Ar. In preferred embodiments, R⁴ is unsubstituted Ar. Most preferably, R⁴ is phenyl.

Further embodiments of the invention relate to compounds of Formula IV wherein X is (CH₂)ₙ. Preferably n is 1-3; most preferably n is 1.

In a preferred embodiment of the present invention, compounds of Formula IV include those in which at least one of R¹, R² and R³ is OH, more preferably at least two of R¹, R² and R³ are OH, while R⁴ is Ar and n is 1-3.

Compounds useful in the compositions and methods disclosed herein include compounds of Formula V and salts, solvates and hydrates thereof: 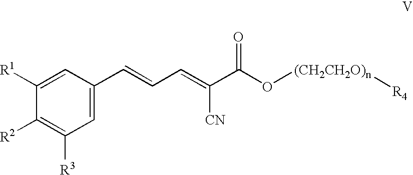
wherein
R¹, R², R³ are each independently selected from H, OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, NO₂, CF₃, OCF₃ and halo;
R⁴═C₁₋₆alkyl; and
n=1-4.

In embodiments of the invention, compounds of Formula V are those in which R¹, R² and R³ are each independently selected from H, OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, NO₂, CF₃, OCF₃ and halo. In preferred embodiments, R¹, R² and R³ are each independently selected from H, OH, C₁₋₄alkyl, C₁₋₄alkoxy, NH₂, NH—C₁₋₄alkyl, N(C₁₋₄alkyl)(C₁₋₄alkyl), NO₂, CF₃, OCF₃ and halo. In more preferred embodiments, R¹, R² and R³ are each independently selected from H, OH, OCH₃, NH₂, N(CH₃)₂, and NO₂. In the most preferred embodiments, R¹, R² and R³ are each independently selected from H, OH and OCH₃.

Further embodiments of the invention relate to compounds of Formula V wherein R⁴ is C₁₋₆alkyl. In preferred embodiments, R⁴ is methyl or ethyl. Most preferably, R⁴ is methyl.

Further embodiments of the invention relate to compounds of Formula V wherein n in 1-4. Preferably, n is 2-3; most preferably, n is 3.

Specific embodiments of the present invention contemplate the use of:
2-Cyano-5-(4-hydroxy-3,5-dimethoxyphenyl)-penta-2E,4E-dienoic acid benzyl ester (CRIX-38)
2-Cyano-5-(3,4-dihydroxyphenyl)-penta-2E,4E-dienoic acid benzyl ester (CRIX-39)
2-Cyano-5-(3,4-dihydroxyphenyl)-penta-2E,4E-dienoic acid 2-[2-(2-methoxyethoxy)ethoxy)]ethyl ester (CRIV-42)
2-Cyano-5-(4-hydroxy-3,5-dimethoxyphenyl)-penta-2E,4E-dienoic acid 2-[2-(2-methoxyethoxy)ethoxy]ethyl ester (CRIV-46); and
2-Cyano-5-(4-hydroxy-3-methoxyphenyl)-penta-2E,4E-dienoic acid benzyl ester (CRIX-79).

In further embodiments, compounds useful in the compositions and methods disclosed herein include compounds of Formula VI, and salts, solvates or hydrates thereof: 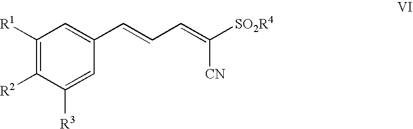
wherein
R¹, R² and R³ are each independently selected from H, OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, NO₂, CF₃, OCF₃ and halo; and
R⁴ is selected from C₁₋₆alkyl, phenyl and pyridyl, wherein phenyl and pyridyl are unsubstituted or substituted with 1-4 substituents, independently selected from C₁₋₆alkyl, C₁₋₆alkoxy and halo.

In embodiments of the invention, compounds of Formula VI are those in which R¹, R² and R³ are each independently selected from H, OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, NO₂, CF₃, OCF₃ and halo. In preferred embodiments, R¹, R² and R³ are each independently selected from H, OH, C₁₋₄alkyl, C₁₋₄alkoxy, NH₂, NH—C₁₋₄alkyl, N(C₁₋₄alkyl)(C₁₋₄alkyl), NO₂, CF₃, OCF₃ and halo. In more preferred embodiments, R¹, R² and R³ are each independently selected from H, OH, OCH₃, NH₂, N(CH₃)₂, N(CH₃)₂ and NO₂. In the most preferred embodiments, R¹, R² and R³ are each independently selected from H, OH and OCH₃.

Further embodiments of the invention relate to compounds of Formula VI wherein R⁴ is selected from C₁₋₆alkyl, phenyl and pyridyl, wherein phenyl and pyridyl are unsubstituted or substituted with 1-4 substituents, independently selected from C₁₋₆alkyl, C₁₋₆alkoxy and halo. In preferred embodiments of the present invention, R⁴ is selected from C₁₋₄alkyl, phenyl and pyridyl, wherein phenyl and pyridyl are unsubstituted or substituted with 1-3 substituents, independently selected from C₁₋₄alkyl, C₁₋₄alkoxy and halo. In more preferred embodiments, R⁴ is selected from CH₃ and phenyl, wherein phenyl is unsubstituted or substituted with 1-2 substituents, independently selected from C₁₋₄alkyl, C₁₋₄alkoxy and halo. In the most preferred embodiment, R⁴ is unsubstituted phenyl.

In a preferred embodiment of the present invention, compounds of Formula IV include those in which at least one of R¹, R² and R³ is OH, more preferably at least two of R¹, R² and R³ are OH, while R⁴ is selected from unsubstituted phenyl and phenyl substituted with 1-4 substituents, independently selected from C₁₋₆alkyl, C₁₋₆alkoxy and halo.

In specific embodiments of the present invention, contemplated compounds include:
2-Benzenesulfonyl-5-(3,4-dihydroxyphenyl)-penta-2E,4E-dienenitrile (CRVIII-33)
2-Benzenesulfonyl-5-(4-hydroxy-3,5-dimethoxyphenyl)-penta-2E,4E-dienenitrile (CRVIII-34)
2-Benzenesulfonyl-5-(4-nitrophenyl)-penta-2E,4E-dienenitrile (CRVIII-35)
5-(3,4-Dihydroxyphenyl)-2-(pyridine-2-sulfonyl)-penta-2E,4E-dienenitrile (CRVIII-50)
2-(4-Chlorobenzenesulfonyl)-5-(3,4-dihydroxyphenyl)-penta-2E,4E-dienenitrile (CRVIII-51)
5-(3,4-Dihydroxyphenyl)2-(toluene-4-sulfonyl)-penta-2E,4E-dienenitrile (CRVIII-52); and
5-(3,4-Dihydroxyphenyl)-2-methanesulfonyl-penta-2E,4E-dienenitrile (CRVIII-53).

The present invention also contemplates the use of prodrugs of the compounds described above. In general, such prodrugs will be functional derivatives of a compound of the invention which are readily convertible in vivo into the compound from which it is notionally derived. Conventional procedures for the selection and preparation of suitable prodrugs are described, for example, in “Design of Prodrugs” ed. H. Bundgaard, Elsevier, 1985.

The present invention also contemplates using radiolabeled forms of compounds of the invention, for example, compounds of the invention labeled by incorporation within the structure ³H or ¹⁴C or a radioactive halogen such as ¹²⁵I.

Compounds useful in the present invention may be prepared by the methods disclosed in WO 03/062190 and PCT CA01/00004.

The compounds may be used in the form of the free base, or in other forms such as salts, prodrugs, solvates, and hydrates, and reference to the formulae provided herein, and CR4, CR11, and CR19 specifically, are intended to encompass all such forms of the compound. The acids which can be used to prepare acid addition salts are those which produce, when combined with the compound, pharmaceutically acceptable salts; that is, salts whose anions are non-toxic to the animal in pharmaceutical doses of the salts, so that the beneficial properties inherent in the free base are not vitiated by side effects ascribable to the anions. Pharmaceutically acceptable salts include those derived from the following acids; mineral acids such as hydrochloric acid, sulfuric acid, phosphoric acid and sulfamic acid; and organic acids such as acetic acid, citric acid, lactic acid, tartaric acid, malonic acid, methanesulfonic acid, ethanesulfonic acid, benzenesulfonic acid, p-toluenesulfonic acid, cyclohexysulfamic acid, quinic acid, and the like.

Similarly, basic addition salt may be prepared using an inorganic base such as lithium, sodium, potassium, calcium, magnesium or barium hydroxide. Illustrative organic bases which form suitable salts include aliphatic, alicyclic or aromatic organic amines such as methylamine, trimethylamine and picoline or ammonia.

The selection of other pharmaceutically acceptable salts will be known to a person skilled in the art and a desired salt may be prepared using standard techniques.

Prodrugs of the compounds may be conventional esters formed with available hydroxy, amino or carboxyl group on the compound. For example, an OH group may be acylated using an activated acid in the presence of a base, and optionally, in inert solvent (e.g. and acid chloride in pyridine). Some common esters which have been utilized as prodrugs are phenyl esters, aliphatic (C₈-C₂₄) esters, acyloxymethyl esters, carbamates and amino acid esters. Conventional procedures for the selection and preparation of suitable prodrugs are described, for example, in “Design of Prodrugs” ed. H. Bundagaard, Elsevier, 1985.

A “solvate” is formed when a suitable solvent are incorporated in the crystal lattice of the compound or salt thereof. A suitable solvent is physiologically tolerable at the dosage administered. Examples of suitable solvents are ethanol, water and the like. When water is the solvent, the molecule is referred to as a “hydrate”. Methods to prepare a solvate are known in the art. In general, solvates are prepared by dissolving the compound in the appropriate solvent and isolating the solvate by cooling or using an antisolvent. The solvent is typically dried or azeotroped under ambient conditions.

The compounds may be administered alone or in combination with a pharmaceutically acceptable carrier, the proportion of which is determined by the solubility and chemical nature of the compound, chosen route of administration and standard pharmaceutical practice.

Preferably the compounds are formulated into pharmaceutical compositions in a biologically compatible form suitable for administration in vivo. Accordingly, in one embodiment, one or more compounds as described above are administered to a human patient in combination with a pharmaceutically acceptable carrier.

The compositions containing the compounds can be prepared by known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the substances in association with one or more pharmaceutically acceptable vehicles or diluents, and contained in buffer solutions with a suitable pH and iso-osmotic with the physiological fluids.

The compounds may be administered to a patient in a variety of forms depending on the selected route of administration, as will be understood by those skilled in the art. The compositions of the invention may be administered orally or parenterally. Parenteral administration includes intravenous, intraperitoneal, subcutaneous, intramuscular, transepithelial, nasal, intrapulmonary, intrathecal, rectal and topical modes of administration. Parenteral administration may be by continuous infusion over a selected period of time.

The compounds may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the compound of the invention may be incorporated with excipient and used in the form of ingestible tablets, buccal tablets, troches, capsules, elixirs, suspensions, syrups, wafers and the like.

The compounds may also be administered parenterally or intraperitoneally. Solutions of a compound can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. Conventional procedures and ingredients for the selection and preparation of suitable formulations are described, for example, in Remington's Pharmaceutical Sciences (1990-18^{th} edition) and in The United States Pharmacopeia: The National Formulary (USP 24 NF19) published in 1999.

The pharmaceutical forms suitable for injectable use include sterile aqueous solutions or dispersion and sterile powders for the extemporaneous preparation of sterile injectable solutions or dispersions. In all cases the form must be sterile and must be fluid to the extent that easy syringeability exists.

An effective amount of the compounds refers to the amount sufficient to inhibit secretion or effects of VEGF, or in the case of treatment of a disorder related to VEGF, the amount sufficient to alleviate, improve, mitigate, ameliorate or cure the disorder or one or more symptoms of the disorder. The clinical effects resulting from inhibition of VEGF secretion or effects of VEGF or treatment of a disorder related to VEGF may be assessed in the known manner, for example, in the case of effect on tumour growth, by tumour shrinkage. The effective amount can vary depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the animal to be treated. One of skill in the art can determine the appropriate dosage based on the above factors. The compounds may be administered initially in a suitable dosage that may be adjusted as required, depending on the clinical response.

The compounds may be packaged as a kit and the invention in one aspect provides a kit or packaged pharmaceutical comprising one or more compounds as disclosed above and instructions or label for use of the compound, including to inhibit secretion or effects of VEGF or to treat a disorder related to VEGF.

The VEGF inhibitors may be provided in sustained release compositions, such as those described in, for example, U.S. Pat. Nos. 5,672,659 and 5,595,760. The use of immediate or sustained release compositions depends on the nature of the condition being treated. If the condition consists of an acute or over-acute disorder, treatment with an immediate release form will be preferred over a prolonged release composition. Alternatively, for certain preventative or long-term treatments, a sustained released composition may be appropriate.

For ocular disorders, the VEGF inhibitor may also be delivered using an intraocular implant. Such implants may be biodegradable and/or biocompatible implants, or may be non-biodegradable implants. The implants may be permeable or impermeable to the active agent, and may be inserted into a chamber of the eye, such as the anterior or posterior chambers or may be implanted in the sclera, transchoroidal space, or an avascularized region exterior to the vitreous. In a preferred embodiment, the implant may be positioned over an avascular region, such as on the sclera, so as to allow for transcleral diffusion of the drug to the desired site of treatment, e.g. the intraocular space and macula of the eye. Furthermore, the site of transcleral diffusion is preferably in proximity to the macula.

Examples of implants for delivery of a VEGF inhibitor include, but are not limited to, the devices described in U.S. Pat. Nos. 3,416,530; 3,828,777; 4,014,335; 4,300,557; 4,327,725; 4,853,224; 4,946,450; 4,997,652; 5,147,647; 5,164,188; 5,178,635; 5,300,114; 5,322,691; 5,403,901; 5,443,505; 5,466,466; 5,476,511; 5,516,522; 5,632,984; 5,679,666; 5,710,165; 5,725,493; 5,743,274; 5,766,242; 5,766,619; 5,770,592; 5,773,019; 5,824,072; 5,824,073; 5,830,173; 5,836,935; 5,869,079, 5,902,598; 5,904,144; 5,916,584; 6,001,386; 6,074,661; 6,110,485; 6,126,687; 6,146,366; 6,251,090; and 6,299,895, and in WO 01/30323 and WO 01/28474, all of which are incorporated herein by reference.

The present invention provides a system comprising a coated medical device, the coating of which is suitable for sustained release of the subject VEGF inhibitor in the locality of the implanted device. Exemplary embodiments are described using an intraluminal medical device, particularly a stent, but the inventive system is also readily applicable to and advantageous in other forms of medical devices.

Once administered, the system remains in the body and serves as a continuous source of the VEGF inhibitor to the affected are& The system according to the present invention permits prolonged release of VEGF inhibitor(s) over a specific period of days, weeks, months (e.g., about 3 months to about 6 months) or years (e.g., about 1 year to about 20 years, such as from about 5 years to about 10 years) until the drug reservoir is used up.

In certain embodiments, the present invention provides an intraluminal medical device for implantation into a lumen of a blood vessel, in particular adjacent an intraluminal lesion such as an atherosclerotic lesion, for maintaining patency of the vessel. In particular embodiments, the present invention provides an elongate radially expandable tubular stent having an interior luminal surface and an opposite exterior surface extending along a longitudinal stent axis, the stent having a coating on at least a portion of the interior or exterior surface thereof. The local delivery of VEGF inhibitor from a stent has the following advantages; namely, the prevention of vessel recoil and remodeling through the scaffolding action of the stent and the prevention of multiple components of neointimal hyperplasia or restenosis as well as a reduction in inflammation and thrombosis. This local administration of VEGF inhibitors to stented coronary arteries may also have additional therapeutic benefit. For example, higher tissue concentrations of the VEGF inhibitor may be achieved utilizing local delivery, rather than systemic administration. In addition, reduced systemic toxicity may be achieved utilizing local delivery rather than systemic administration while maintaining higher tissue concentrations. Also in utilizing local delivery from a stent rather than systemic administration, a single procedure may suffice with better patient compliance.

There are a multiplicity of different stents that may be utilized following percutaneous transluminal coronary angioplasty. Although any number of stents may be utilized in accordance with the present invention, for simplicity, a limited number of stents will be described in exemplary embodiments of the present invention. The skilled artisan will recognize that any number of stents may be utilized in connection with the present invention.

In addition, as stated above, other medical devices may be utilized. In other embodiments according to the present invention, the polymer in which a sustained release VEGF inhibitor formulation is suspended or dispersed is coated onto a surgical implement such as surgical tubing (such as colostomy, peritoneal lavage, catheter, and intravenous tubing). In still further embodiments according to the present invention, the device is an intravenous needle having the polymer and a corticosteroid (or codrug or prodrug thereof) coated thereon.

A stent is commonly used as a tubular structure left inside the lumen of a duct to relieve an obstruction. Commonly, stents are inserted into the lumen in a non-expanded form and are then expanded autonomously, or with the aid of a second device in situ. A typical method of expansion occurs through the use of a catheter-mounted angioplasty balloon which is inflated within the stenosed vessel or body passageway in order to shear and disrupt the obstructions associated with the wall components of the vessel and to obtain an enlarged lumen.

The stents of the present invention may be fabricated utilizing any number of methods. For example, the stent may be fabricated from a hollow or formed stainless steel tube that may be machined using lasers, electric discharge milling, chemical etching or other means. The stent is inserted into the body and placed at the desired site in an unexpanded form. In one exemplary embodiment, expansion may be effected in a blood vessel by a balloon catheter, where the final diameter of the stent is a function of the diameter of the balloon catheter used.

It should be appreciated that a stent in accordance with the present invention may be embodied in a shape-memory material, including, for example, an appropriate alloy of nickel and titanium or stainless steel.

Structures formed from stainless steel may be made self-expanding by configuring the stainless steel in a predetermined manner, for example, by twisting it into a braided configuration. In this embodiment after the stent has been formed it may be compressed so as to occupy a space sufficiently small as to permit its insertion in a blood vessel or other tissue by insertion means, wherein the insertion means include a suitable catheter, or flexible rod.

On emerging from the catheter, the stent may be configured to expand into the desired configuration where the expansion is automatic or triggered by a change in pressure, temperature or electrical stimulation.

Regardless of the design of the stent, it is preferable to have the sustained release VEGF inhibitor formulation applied with enough specificity and a sufficient concentration to provide an effective dosage in the lesion area. In this regard, the “reservoir size” in the coating is preferably sized to adequately apply the VEGF inhibitor formulation at the desired location and in the desired amount and for a sustained period of time.

Various embodiments of this invention comprise polymers with varied physical characteristics. In some embodiments according to the invention, the system comprises a polymer that is relatively rigid. In other embodiments, the system comprises a polymer that is soft and malleable. In still other embodiments, the system includes a polymer that has an adhesive character. Hardness, elasticity, adhesive, and other characteristics of the polymer may be varied as necessary.

Any number of bioerodible or non-erodible polymers may be utilized in conjunction with the VEGF inhibitors. Polymers may be advantageously selected from among those which reduce the rate of diffusion of the VEGF inhibitor. Polymers that can be used for coatings in this application can be absorbable or non-absorbable and must be biocompatible to minimize irritation to the vessel wall. The polymer may be either biostable or bioabsorbable depending on the desired rate of release or the desired degree of polymer stability, but a bioabsorbable polymer may be preferred since, unlike biostable polymer, it will not be present long after implantation to cause any adverse, chronic local response.

In some embodiments according to the present invention, the polymer coating is permeable to water in the surrounding tissue, e.g. in blood plasma. In such cases, water solution may permeate the polymer, thereby contacting the VEGF inhibitor. The rate of dissolution may be governed by a complex set of variables, such as the polymer's permeability, the solubility of the VEGF inhibitor, the pH, ionic strength, and protein composition, etc. of the physiologic fluid. In certain embodiments, however the permeability may be adjusted so that the rate of dissolution is governed primarily, or in some cases practically entirely, by the solubility of the VEGF inhibitor in the ambient liquid phase. In still other embodiments the VEGF inhibitor may have a high solubility in the surrounding fluid. In such cases the matrix permeability may be adjusted so that the rate of dissolution is governed primarily, or in some cases practically entirely, by the permeability of the polymer.

Suitable bioerodible and bioabsorbable polymers that could be used include polymers selected from the group consisting of aliphatic polyesters, poly(amino acids), copoly(ether-esters), polyalkylenes oxalates, polyamides, poly(iminocarbonates), polyorthoesters, polyoxaesters, polyamidoesters, polyoxaesters containing amido groups, poly(anhydrides), polyphosphazenes, biomolecules and blends thereof. For the purpose of this invention aliphatic polyesters include homopolymers and copolymers of lactide (which includes lactic acid d-,l- and meso lactide), E-caprolactone, glycolide (including glycolic acid), hydroxybutyrate, hydroxyvalerate, para-dioxanone, trimethylene carbonate (and its alkyl derivatives), 1,4-ioxepan-2-one, 1,5-dioxepan-2-one, 6,6-dimethyl-1,4-dioxan-2-one and polymer blends thereof. Poly(iminocarbonate) for the purpose of this invention include as described by Kemnitzer and Kohn, in the Handbook of Biodegradable Polymers, edited by Domb, Kost and Wisemen, Hardwood Academic Press, 1997, pages 251-272. Copoly(ether-esters) for the purpose of this invention include those copolyester-ethers described in Journal of Biomaterials Research, Vol. 22, pages 993-1009, 1988 by Cohn and Younes and Cohn, Polymer Preprints (ACS Division of Polymer Chemistry) Vol. 30(1), page 498, 1989 (e.g. PEO/PLA). Polyalkylene oxalates for the purpose of this invention include U.S. Pat. Nos. 4,208,511; 4,141,087; 4,130,639; 4,140,678; 4,105,034; and 4,205,399 (incorporated by reference herein). Polyphosphazenes, co-, ter- and higher order mixed monomer based polymers made from L-lactide, D,L-lactide, lactic acid, glycolide, glycolic acid, para-dioxanone, trimethylene carbonate and □-caprolactone such as are described by Allcock in The Encyclopedia of Polymer Science, Vol. 13, pages 31-41, Wiley Intersciences, John Wiley & Sons, 1988 and by Vandorpe, Schacht, Dejardin and Lemmouchi in the Handbook of Biodegradable Polymers, edited by Domb, Kost and Wisemen, Hardwood Academic Press, 1997, pages 161-182 (which are hereby incorporated by reference herein). Polyanhydrides from diacids of the form HOOC—C6H4-O—(CH2)m-O—C6H4-COOH where m is an integer in the range of from 2 to 8 and copolymers thereof with aliphatic alpha-omega diacids of up to 12 carbons. Polyoxaesters polyoxaamides and polyoxaesters containing amines and/or amido groups are described in one or more of the following U.S. Pat. Nos. 5,464,929; 5,595,751; 5,597,579; 5,607,687; 5,618,552; 5,620,698; 5,645,850; 5,648,088; 5,698,213 and 5,700,583; (which are incorporated herein by reference). Polyorthoesters such as those described by Heller in Handbook of Biodegradable Polymers, edited by Domb, Kost and Wisemen, Hardwood Academic Press, 1997, pages 99-118 (hereby incorporated herein by reference).

Moreover, suitable polymers include naturally occurring or synthetic materials that are biologically compatible with bodily fluid and mammalian tissues. Polymeric biomolecules for the purpose of this invention include naturally occurring materials that may be enzymatically degraded in the human body or are hydrolytically unstable in the human body such as fibrin, fibrinogen, collagen, elastin, and absorbable biocompatible polysaccharides such as chitosan, starch, fatty acids (and esters thereof), glucoso-glycans and hyaluronic acid.

The VEGF inhibitor can also be administered in combination with any other method of treatment of the particular disorder. For instance, the VEGF inhibitor can be administered in combination with one or more suitable adjuvants, such as, e.g., cytokines or other immune stimulators. In other embodiments, the VEGF inhibitor is administered with an anti-inflammatory drug, such as an NSAIDs or other agent that exerts anti-inflammatory, analgesic and antipyretic activity. These include salicylates such as aspirin, sodium salicylate, choline salicylate, salicylsalicylic acid, diflunisal, aloxiprine, lysine-acetyl salicylate, benorilate, calcium carcasalate, and salsalate; indoleacetic acids such as indomethacin and proglumethacin; aryl-acetic acids such as bufexamac, diclofenac, tolmetin and sulindac; pyrazoles such as phenylbutazone, oxyphenbutazone; pynrolealklanoic acids such as tolmetin; phenylacetic acids such as ibuprofen, feroprofen, flurbiprofen, and ketoprofen; fenamates such as nifluminic acid, mefanamic acid, and meclofenainate; oxicams such as piroxicarn and tenoxicarn; naphthaleneacetic acids such as naproxen; and gold salts such as sodium aurothiopropanolsulphonate and auranofin. Adrenal corticosteroids are alternatives to NSAIDs for treating inflammatory diseases. These steroids include hydrocortisone, prednisolone, methylprednisolone, triamicinolone, dexamethasone and betaroethasone.

All references cited herein are fully incorporated by reference.

### EXAMPLES

The compounds CR4, CR11 and CR19 may be prepared as described in Examples 1 to 14.

### Materials and Methods for Examples 1-14

¹H NMR spectra were obtained on a Varian Unity Plus spectrometer (USA) at 500 MHz with tetramethylsilane (TMS, Me₄Si) as an internal standard (δ=0). Electrospray mass spectra were recorded on an API III Plus triple quadrupole mass spectrometer (USA), with a direct introduction of the samples into the ionization source. Thin layer chromatography was performed with UV-254 aluminum-backed TLC sheets of 0.25 mm thickness (Kieselgel 60 F₂₅₄, Merck, Germany). HPLC separation of the compound of Example 13 was performed on a Waters 600 chromatograph (USA), column Nova-Pak C18 3.9×306 mm (Waters, USA). Vacuum distillations were done using Kugelrohr apparatus (Aldrich, USA) at stated temperatures of an oven. 3,5-Dimethoxy-4-hydroxycinnamaldehyde, 3,4-dimethoxycinnamic acid, 3,4-dihydroxycinnamic acid, 3,4-dimethoxybenzylamine, benzylamine, methyl cyanoacetate, were purchased from Aldrich (USA) and were used as received. The reagents were from Aldrich (USA). Solvents were purchased from Caledon (Canada).

### Example 1

### N-(Cyanoacetyl)3,4-dimethoxybenzylamide (A₁)

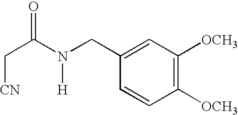

To 3,4-dimethoxybenzylamine (2.7 ml, 18 mmol) methyl cyanoacetate was added (1.6 ml, 18 mmol). The reaction was heated for 14 h at 100° C. Cooling gave a dark brown solid which was recrystallized from ethanol to give 2.90 g of the product (69% yield).

The product gave the following analytical data:

NMR (CD₃COCD₃, δ, ppm): 3.62 (s, 2H, CH₂CN), 3.78 (s, 6H, (OMe)₂), 4.34 (br.s., 2H, NHCH₂Ph), 6.84 (dd, 1H, J 1.95 and 8.1 Hz, H⁶), 6.88 (d, 1H, J 8.1 Hz, H⁵), 6.93 (d, 1H, J 1.95 Hz, H²), 7.80 (br.s., 1H, NH).

MS, m/e (rel. intensity, %): 235 (19) [M+H]⁺, 252 (100) [M+NH₄]⁺, 257 (33) [M+Na]⁺.

### Example 2

### N-(Cyanoacetyl)3,4-dihydroxybenzylamide (A₂)

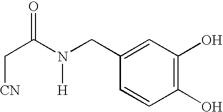

To N-(cyanoacetyl)3,4-dimethoxybenzylamide (Example 1, 0.2 g, 0.85 mmol) in 20 ml of CH₂Cl₂ boron tribromide was added under argon at −78° C. (0.24 ml, 2.56 mmol) in 2.5 ml of CH₂Cl₂. After 2 h the reaction was brought to room temperature and stirred overnight. The reaction was cooled to 0° C., 10 ml of 1N HCl was added, the solution was extracted with 3×50 ml of ethyl acetate, the organic phase was washed to neutral pH, dried with MgSO₄, and taken to dryness. The residue was purified by silica gel chromatography (CHCl₃-MeOH, 20:1) to give a yellow solid (0.07 g, 40% yield). The product gave the following analytical data:

NMR (CD₃COCD₃, δ, ppm): 2.83 (s, (OH)₂), 3.60 (s, 2H, CH₂CN), 4.25 (br.s., 2H, NHCH₂Ph), 6.63 (dd, 1H, J 1.95 and 8.1 Hz, H⁶), 6.75 (d, 1H, J 8.1 Hz, H⁵), 6.79 (d, 1H, J 1.95 Hz, H²), 7.71 (br.s., 1H, NH).

MS, m/e (rel. intensity, %): 207 (38) [M+H]⁺, 224 (100) [M+NH₄]⁺, 229 (2.6) [M+Na]⁺.

### Example 3

### (E,E)-2-(3,4-Dihydroxybenzylaminocarbonyl)-3-(3,5-dimethoxy-4-hydroxystyryl)acrylonitrile (CR11)

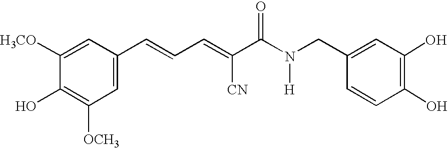

To 3,5-dimethoxy-4-hydroxycinnamaldehyde (0.042 g, 0.2 mmol) and N-(cyanoacetyl)3,4-dihydroxybenzylamide (Example 2, 0.042 g, 0.2 mmol) in 10 ml of ethanol 3 mg of β-alanine was added and the reaction was refluxed for 6 h. Water was added and the solid was recrystallized from 5 ml of ethanol twice to give 0.06 g (75%) of a red solid The product gave the following analytical data:

NMR (CD₃COCD₃, δ, ppm): 2.81 (s, (OH)₃), 3.89 (s, 6H, (OMe)₂), 4.39 (br.s., 2H, NHCH₂Ph), 6.68 (dd, 1H, J 1.95 and 8.1 Hz, H^{6′}), 6.76 (d, 1H, J 8.1 Hz, H^{5′}), 6.86 (d, 1H, J 1.95 Hz, H^{2′}), 7.07 (br.s, 2H, H²⁺⁶), 7.16 (dd, 1H, J 11.7 and 15.1 Hz, PhCCHCCN olefinic), 7.37 (d, 1H, J 15.1 Hz, PhCH olefinic), 7.70 (br.s., 1H, NH), 7.98 (dd, 1H, J 0.75 and 11.7 Hz, CHCN olefinic).

MS, m/e (rel. intensity, %): 397 (100) [M+H]⁺, 414 (14) [M+NH₄]⁺.

### Example 4

### N-(Cyanoacetyl)benzylamide (A₃)

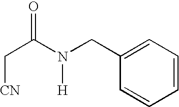

The compound was prepared as described in Example I by adding methyl cyanoacetate (1.3 ml, 14 mmol) to benzylamine (1.5 ml, 14 mmol). The compound was distilled in vacuo directly from the reaction mixture (Kugelrohr apparatus (Aldrich), 0.1 mm Hg, T. oven 180-190° C.) to give an off-white solid (2.34 g, 95%). The product gave the following analytical data:

NMR (CD₃COCD₃, δ, ppm): 3.39 (s, 2H, CNCH₂), 4.46 (d, 2H, J 5.4 Hz, NHCH₂Ph), 6.40 (br.s., 1H, NH), 7.24-7.36 (m, 5H, Ph).

MS, m/e (rel. intensity, %): 175 (64) [M+H]⁺, 192 [M+NH₄]⁺.

### Example 5

### 3,4-Dimethoxycinnamyl alcohol (A₆)

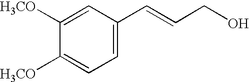

To a solution of 0.42 g (2.0 mmol) of 3,4-dimethoxycinnamic acid in 50 ml MeOH was added SOCl₂ (50 μl) and the mixture was stirred at 60° C. for 5 h. Methanol was taken to dryness and the obtained 3,4-dimethoxycinnamic acid methyl ester was reduced with 1M THF solution of diisobutylaluminum hydride (8.0 mmol) in absolute THF (50 ml) at 20° C. for 1 h. Water was added, the mixture was extracted with EtOAc, dried with MgSO₄ and distilled in vacuo (Kugelrohr apparatus (Aldrich), 0.1 mm Hg, T. oven 185-190° C.) giving an off-white solid, yield 0.36 g (92%), m.p. 70-71° C. The product gave the following analytical data:

NMR (CD₃COCD₃, δ, ppm): 3.77, 3.82 (2×s, 2×3H, OMe+OMe), 4.19 (d, 2H, J 5.0 Hz, CH₂OH), 6.25 (dt, 1H, J 5.0 and 15.5 Hz, PhCCH olefinic), 6.51 (d, 1H, J 15.5 Hz, PhCH olefinic), 6.89 (m, 2H, H⁵⁺⁶), 7.05 (br.s., 1H, H²).

MS, m/e (rel. intensity, %): 177 (100) [M-OH]+, 195 (4) [M+H]⁺, 212 (59) [M+NH₄]⁺, 217 (26) [M+Na]⁺.

### Example 6

### 3,4-Dimethoxycinnamaldehyde (A₇)

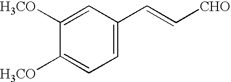

To a mixture of pyridinium dichromate (3.88 g, 10.3 mmol) and 4 g of finely grounded freshly activated molecular sieves 3 Å in 20 ml of CH₂Cl₂ 3,4-dimethoxycinnamyl alcohol in 10 ml of CH₂Cl₂ (Example 5, 1.00 g, 5.1 mmol) was added. The reaction was stirred for 2 h, 0.5 ml of methanol was added, the residue was passed through silica gel and washed with 300 ml of ethyl acetate. After evaporation the compound was purified by silica gel chromatography (hexane-EtOAc, 5:1) leading to a crystallizing oil (0.62 g, 63%).

The product gave the following analytical data:

NMR (CD₃COCD₃, δ, ppm): 3.90 (2×s, 2×3H, OCH₃+OCH₃), 6.70 (dd, 1H, J 7.6 and 16.0 Hz, PhC═CH olefinic), 7.05 (d, 1H, J 8.3 Hz, H⁵), 7.28 (dd, 1H, J 1.4 and 8.3 Hz, H⁶), 7.37 (d, 1H, J 1.4 Hz, H²), 7.60 (d, 1H, J 16.0 Hz, PhCH olefinic), 9.65 (d, 1H, J 7.6 Hz, CHO).

MS, m/e (rel. intensity, %): 193 (100) [M+H]⁺, 210 (26) [M+NH₄]⁺.

### Example 7

### (E,E)-2-(Benzylaminocarbonyl)-3-(3,4-dimethoxystyryl)acrylonitrile (CR2)

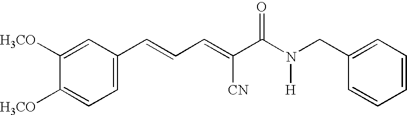

The compound was prepared as described in Example 3, by adding 3,4-dimethoxycinnamaldehyde (Example 6, 0.04 g, 0.2 mmol) to N-(cyanoacetyl)benzylamide (Example 4, 0.036 g, 0.2 mmol). After refluxing for 1 h and recrystallization from ethanol a yellow solid was obtained (0.045 g, 62%). The product gave the following analytical data:

NMR (CD₃COCD₃, δ, ppm): 3.90 (s, 2×3H, OMe+OMe), 4.57 (d, 2H, J<2 Hz, NHCH₂Ph), 7.08 (br.s., 1H, H²), 7.17 (dd, 1H, J 11.5 and 15.2 Hz, PhCCHCCN olefinic), 7.23-7.42 (m, 8H, aromatic+H⁵+H⁶+PhCH olefinic), 7.90 (br.t, 1H, NH), 8.05 (dd, 1H, J 0.55 and 11.5 Hz, CHCN olefinic).

### Example 8

### (E,E)-2-(Benzylaminocarbonyl)-3-(3,4-dihydroxystyryl)acrylonitrile (CR4)—Method A

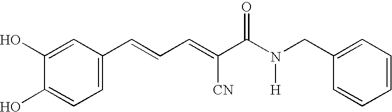

Boron tribromide (0.033 ml, 0.34 mmol) was added to (E,E)-2-(benzylaminocarbonyl)-3-(3,4-dimethoxystyryl)acrylonitrile (Example 7, 0.04 g, 0.11 mmol). The residue was purified by silica gel chromatography (CHCl₃-MeOH, 10:1) to give an orange solid (0.02 g, 55% yield). The product gave the following analytical data:

NMR (CD₃COCD₃, δ, ppm): 2.86 (br.s., 2H, (OH)₂), 4.55 (m, 2H, NHCH₂Ph), 6.90-7.42 (m, 10H, Ph+Ph′+olefinic), 7.87 (br.s., 1H, NH), 8.02 (dd, 1H, J<0.5 and 11.4 Hz, CHCN olefinic).

MS, m/e (rel. intensity, %): 295 (61) [M+H—CN]⁺, 321 (100) [M+H]⁺, 338 (30) [M+NH₄]⁺.

### Example 9

### Methyl ester of 3,4-bis(t-butyldimethylsilyloxy)cinnamic acid (A₈)

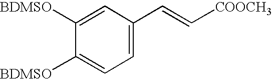

To a solution of 3.6 g (20 mmol) of 3,4-dihydroxycinnamic acid in 300 ml MeOH was added SOCl₂ (100 μl) and the mixture was stirred at 60° C. for 5 h. Methanol was taken to dryness and the obtained methyl ester was treated up with 10.2 g (68 mmol) of t-BuMe₂SiCl and 9.2 g (136 mmol) of imidazole in 100 ml DMF at 50° C. for 0.5 h. Mixture was diluted with water and extracted with hexane. Hexane was taken to dryness. The residue was distilled in vacuo (Kugelrohr apparatus (Aldrich), 0.1 mm Hg, T. oven 200-210° C.) and crystallized from hexane at −20° C. giving a white solid, yield 7.5 g (89%), m.p. 57-58° C. The product gave the following analytical data:

MS, m/e (rel. intensity, %): 423 (100) [M+H]⁺, 440 (98) [M+NH₄]⁺.

### Example 10

### 3,4-Bis(t-butyldimethylsilyloxy)cinnamyl alcohol (A₉)

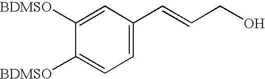

The compound was prepared as described in Example 5 by treating of 3,4-dihydroxycinnamic acid bis(BDMS) ether methyl ester (Example 9, 0.42 g, 1.0 mmol) with 1M THF solution of diisobutylaluminum hydride (4.0 mmol) in absolute THF (25 ml) at 20° C. for 1 h. After distilling in vacuo (Kugelrohr apparatus (Aldrich), 0.1 mm Hg, T. oven 185-200° C.) a white viscous oil was obtained, yield 0.33 g (85%). The product gave the following analytical data:

NMR (CD₃COCD₃, δ, ppm): 0.23, 0.24 (2×s, 2×6H, Me₂Si+Me₂Si), 1.00, 1.02 (2×s, 2×9H, t-BuSi+t-BuSi), 4.19 (d, 2H, J 4.9 Hz, CH₂OH), 6.22 (dt, 1H, J 4.9 and 16.0 Hz, PhCCH olefinic), 6.49 (d, 1H, J 16.0 Hz, PhCH olefinic), 6.85 (d, 1H, J 8.2 Hz, H⁵), 6.92 (dd, 1H, J 2.1 and 8.2 Hz, H⁶), 6.97 (d, 1H, J 2.1 Hz, H²).

MS, m/e (rel. intensity, %): 377 (100) [M-OH]⁺, 395 (2) [M+H]⁺, 412 (15) [M+NH₄]⁺.

### Example 11

### 3,4-Bis(t-butyldimethylsilyloxy)cinnamaldehyde (A₁₀)

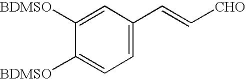

The compound was prepared as described in Example 6 by adding 3,4-bis(t-butyldimethylsilyloxy)cinnamyl alcohol (Example 10, 0.2 g, 0.5 mmol) in 5 ml of CH₂Cl₂ to a mixture of pyridinium dichromate (0.38 g, 1 mmol) and 1 g molecular sieves 3 Å in 20 ml of CH₂Cl₂. The residue was passed through silica gel and washed with 300 ml of EtOAc-hexane, 1:1. After evaporation the compound was purified by silica gel chromatography (hexane-EtOAc, 5:1) leading to an oil (0.15 g, 76%). The product gave the following analytical data:

NMR (CD₃COCD₃, δ, ppm): 0.26 and 0.28 (2×s, 2×6H, Me₂Si+Me₂Si), 1.01 and 1.02 (2×s, 2×9H, t-BuSi+t-BuSi), 6.60 (dd, 1H, J 7.7 and 15.9 Hz, PhCCH olefinic), 7.01 (dd, 1H, J<0.5 and 8.9 Hz, H⁶), 7.27 (m, 2H, H²⁺⁵), 7.60 (d, 1H, J 15.9 Hz, PhCH olefinic), 9.65 (d, 1H, J 7.7 Hz, CHO).

MS, m/e (rel. intensity, %): 367 (3) [M+H—CN]⁺, 393 (100) [M+H]⁺, 410 (10) [M+NH₄]⁺.

### Example 12

### (E,E)-2-(Benzylaminocarbonyl)-3-(3,4-bis(t-butyldimethylsilyloxystyryl))acrylonitrile (CR18)

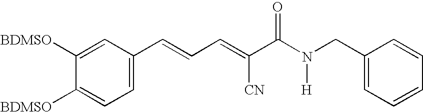

The compound was prepared as described in Example 3 by adding 3,4-bis(t-butyldimethylsilyloxy)cinnamaldehyde (Example 11, 0.100 g, 0.26 mmol) to N-(cyanoacetyl)benzylamide (Example 4, 0.044 g, 0.26 mmol. After refluxing for 2.5 h purification by silica gel chromatography (hexane-EtOAc, 15:1) provided a yellow solid (0.090 g, 64%). The product gave the following analytical data:

NMR (CD₃COCD₃, δ, ppm): 0.24 and 0.25 (2×s, 2×6H, Me₂Si+Me₂Si), 1.01 and 1.02 (2×s, 2×9H, t-BuSi+t-BuSi), 4.55 (br.s., 2H, NHCH₂Ph), 7.00 (d, 1H, J 8.5 Hz, H⁴), 7.12 (dd, 1H, J 11.7 and 15.6 Hz, PhCCHCCN olefinic), 7.24-7.43 (m, 8H, aromatic and olefinic protons), 7.93 (br.s., 1H, NH), 8.02 (dd, 1H, J<0.5 and 11.7 Hz, CHCN olefinic).

MS, m/e (rel. intensity, %): 523 (30) [M+H—CN]⁺, 540 (24) [M+NH₄—CN]⁺, 549 (89) [M+H]⁺, 566 (100) [M+NH₄]⁺.

### Example 13

### (E,E)-24Benzylaminocarbonyl)-3-(3,4-dihydroxystyryl)acrylonitrile (CR4)—Method B

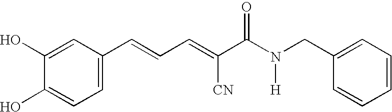

(E,E)-2-Benzylaminocarbonyl-3-[3,4-bis(t-butyldimethylsilyloxystyryl)]acrylonitrile (Example 12, 0.028 g, 0.052 mmol) was treated with 60 μl of a 1M THF solution of tetra-n-butylammonium fluoride in 2 ml of dry THF for 0.5 h at 20° C. After evaporation the compound was dissolved in 5 ml of chloroform-methanol, 20:1, passed through silica gel and washed with chloroform-methanol, 20:1. The residue was purified by HPLC chromatography (MeCN—H₂O, 60:40, UV detection at 340 nm) leading to an orange solid (0.010 g, 62%). The analytical data were identical to the compound prepared as described in Example 8.

### Example 14

### (E,E)-2-(3,4 Dihydroxybenzylaminocarbonyl)-3-styrylacrylonitrile (CR19)

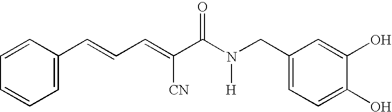

The compound was prepared as described in Example 3 by adding cinnamaldehyde (0.018 ml, 0.14 mmol) to N-(cyanoacetyl)3,4-dihydroxybenzylamide (Example 2, 0.03 g, 0.14 mmol). After refluxing for 2 h and recrystallization from ethanol, a yellow solid was obtained (0.027 g, 59%). The product gave the following analytical data:

NMR (CD₃COCD₃, δ, ppm): 2.82 (br.s., 2H, (OH)₂), 4.39 (br.s., 2H, NHCH₂Ph), 6.70 (dd, 1H, J 1.9 and 8.2 Hz, H^{6′}), 6.76 (d, 1H, J 8.2 Hz, H^{5′}), 6.87 (d, 1H, J 1.9 Hz, H^{2′}), 7.30 (dd, 1H, J 11.3 and 15.7 Hz, PhCCHCCN olefinic), 7.47 and 7.73 (2×m, 6H, aromatic protons and PhCH olefinic), 7.82 (br.s., 1H, NH), 8.04 (dd, 1H, J<0.5 and 11.3 Hz, CHCN olefinic).

MS, m/e (rel. intensity, %): 321 (100) [M+H]⁺, 338 (65) [M+NH₄]⁺.

### Example 15

VEGF secretion from the breast cancer cell lines HTB-133 (KDR+), HTB-131 (KDR−) and MDA-231, HTB-181 prostate cancer cell line, HTB-72 melanoma cell line, CR2-1730 human umbilical vascular endothelial (HUV-EC-C) cell line and normal HUVEC primary cells was measured as follows.

10⁵-10⁶ cells/ml of each of the above cells lines and primary cells were grown for 24 hours in a medium containing 10% FCS which does not contain VEGF. Medium was collected and analysed for the presence of VEGF using VEGF-165 ELISA kit (R&D Systems).

Results: All cell lines tested secreted to the medium 2-3 ng/ml of VEGF. No VEGF secretion was detected in normal HUVEC primary cells.

### Example 16

CR-4 dependent inhibition of VEGF secretion from breast, prostate, melanoma and HUVEC cell lines.

10⁶ cells/ml of each cell lines were incubated for 5 hrs. with various concentrations of CR-4. Cells were washed twice in PBS to remove CR4 and fresh medium was added for 24 hrs. Medium was collected and analysed for the presence of VEGF using VEGF-165 ELISA kit (R&D Systems).

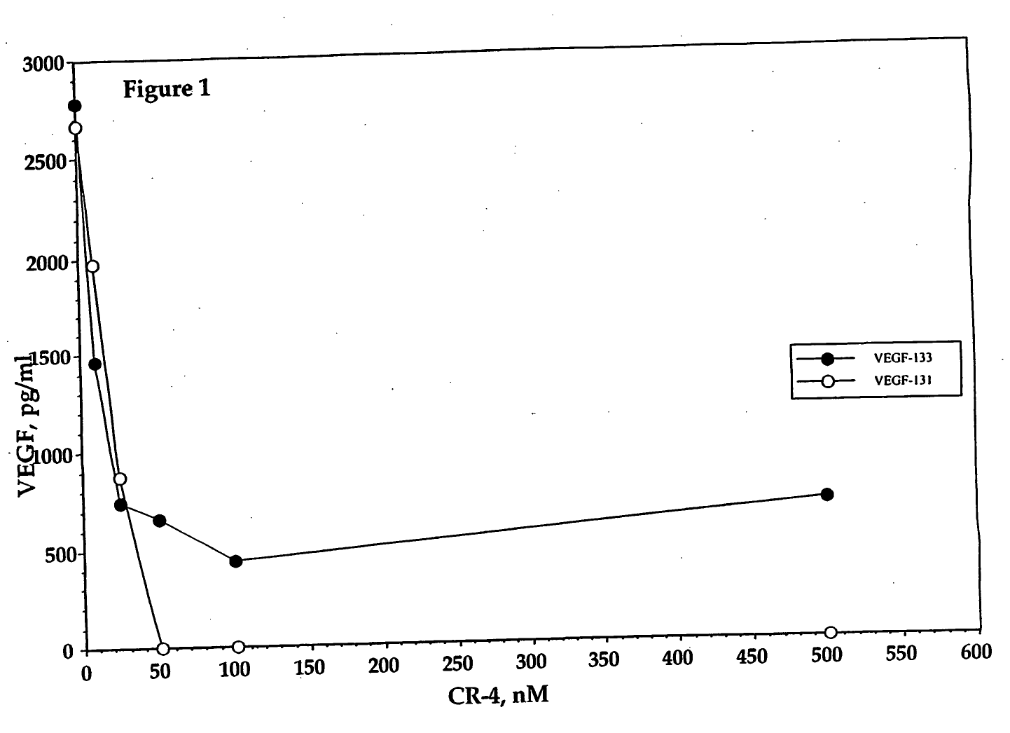

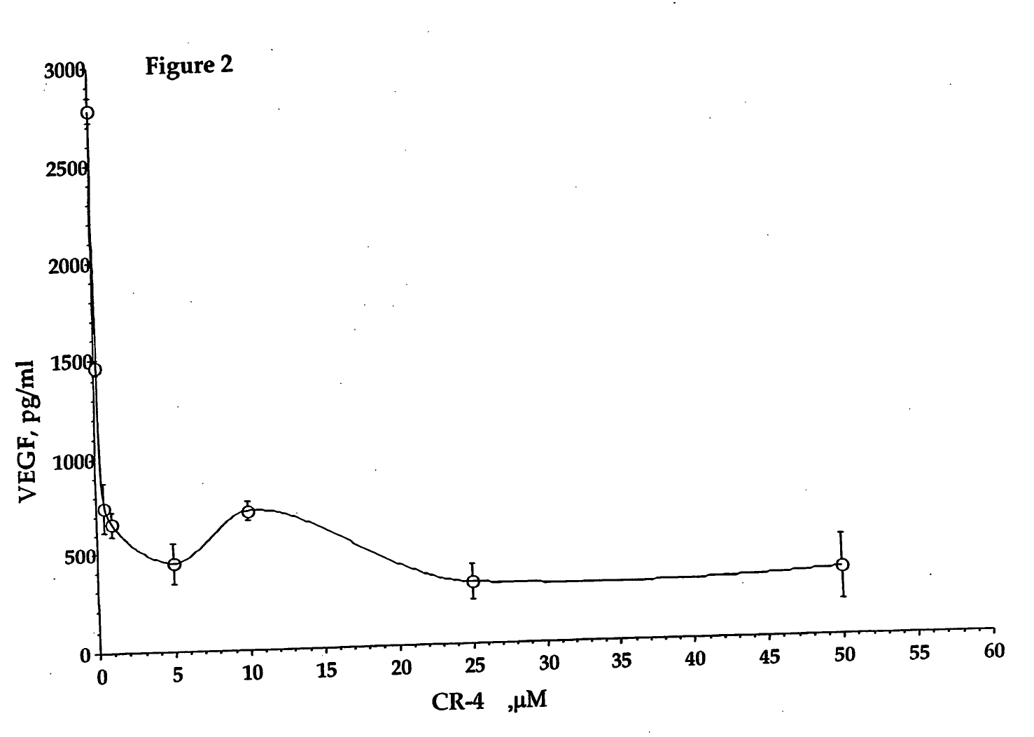

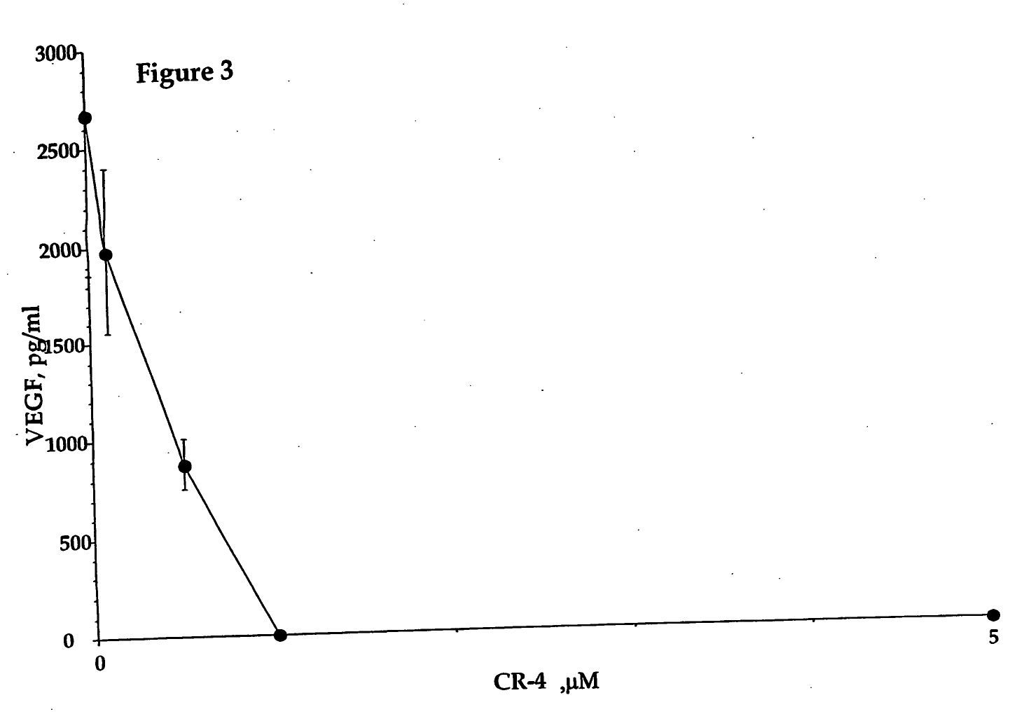

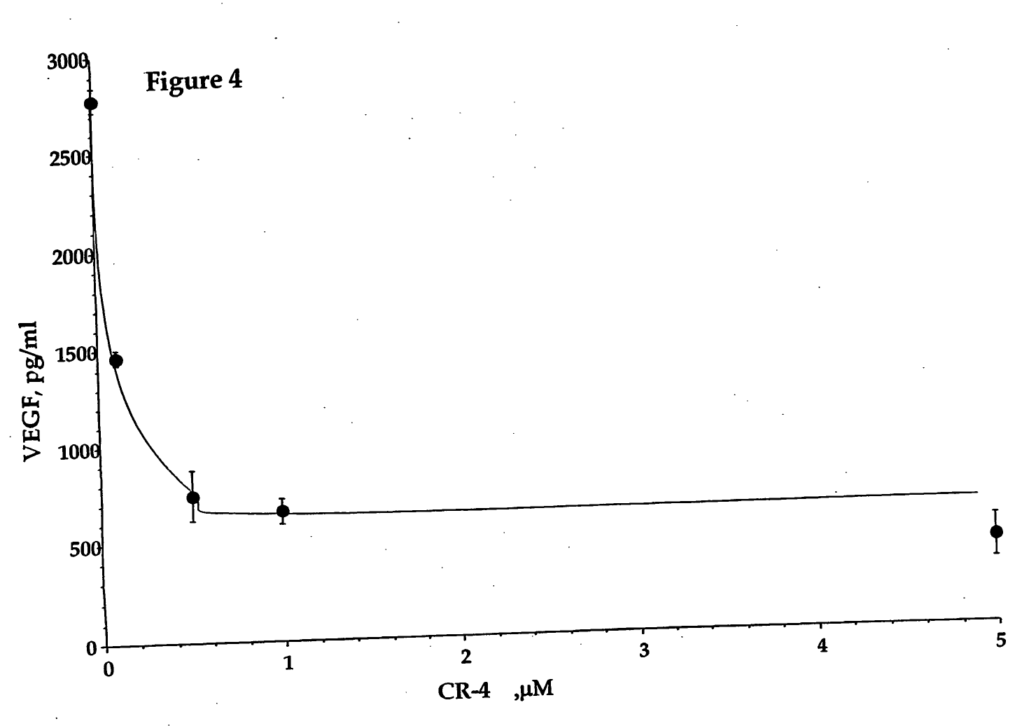

Results: As shown in FIGS. 1 to 4, CR-4 dose dependent inhibition, with an IC50 of 10-30 nM, of VEGF secretion was observed in all cell lines.

### Example 17

CR-4 dependent inhibition of VEGF secretion results in the inhibition of HUVEC growth.

10⁵ cells/ml of KDR⁺ HTB-133 and KDR⁻ HTB-131 breast cancer cell lines were plated and grown over night in HUVEC medium which does not contain VEGF (M199 containing 10% FCS, heparin, antibiotics and endothelial cell growth supplement). After an incubation for 5 hrs. with various concentrations of CR-4 the wells were washed twice and fresh HUVEC medium was added for 24 hrs. This medium with or without 10 ng/ml of recombinant VEGF was then transferred to parallel wells containing 10⁵ cells/ml of HUVEC that were plated and grown in HUVEC medium for 24 hrs. Radioactive labelled [³H]-Thymidine was added to HUVEC wells over night. Cells were harvested and their [³H]-Thymidine incorporation was measured.

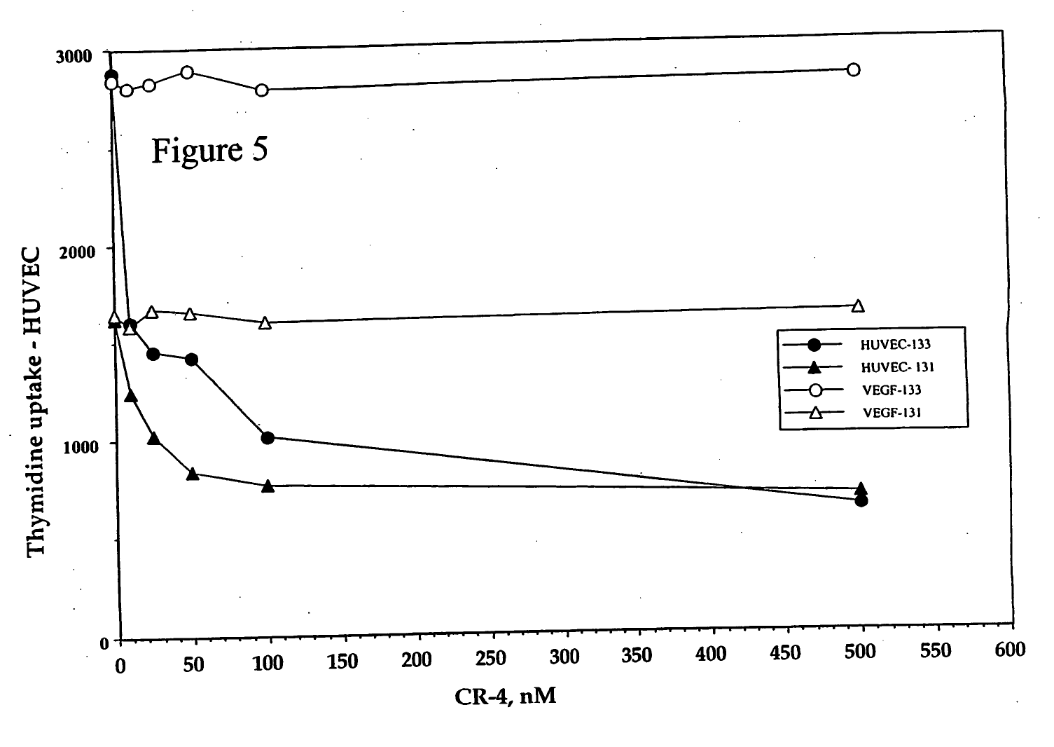

Results: FIG. 5 shows CR-4 dose dependent inhibition, with an IC50 of 20-30 nM, of HUVEC growth is shown. This inhibition is rescued by recombinant VEGF.

### Example 18

CR11 and CR19 dependent inhibition of VEGF secretion from breast cancer cell lines.

Procedure: 10⁶ cells/ml of each of HTB-133 and MDA-231 cells were incubated for 5 hours with various concentrations of CR11 or CR19. Cells were washed twice in PBS to remove CR11 and CR19 and fresh medium was added for 24 hours. Medium was collected and analysed for the presence of VEGF using VEGF-165 ELISA kit (R&D Systems).

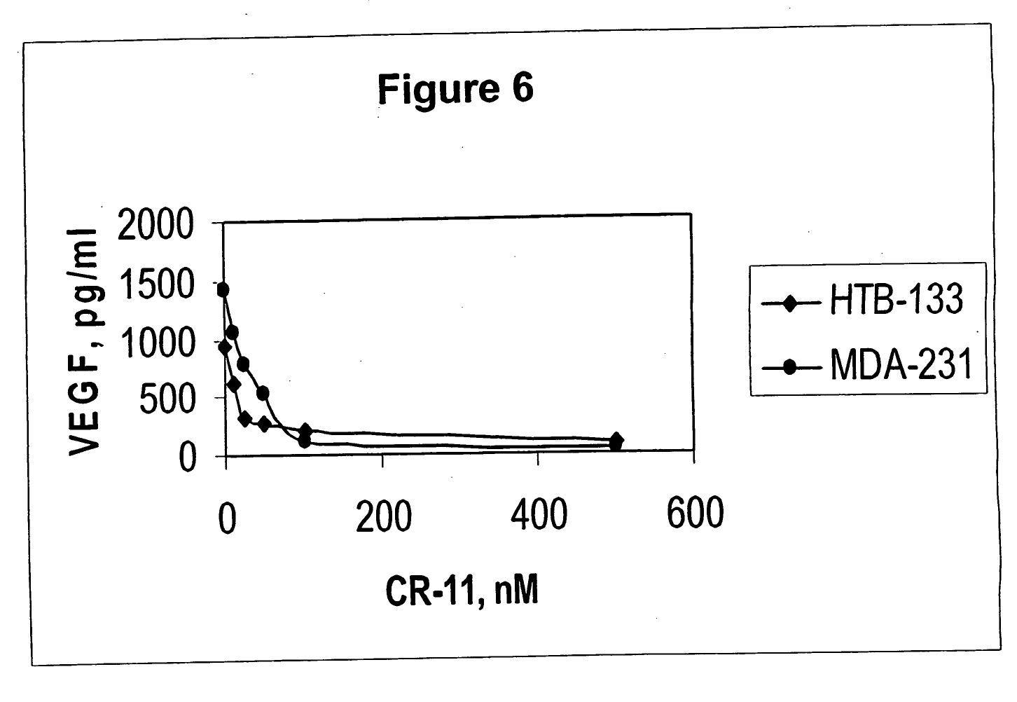

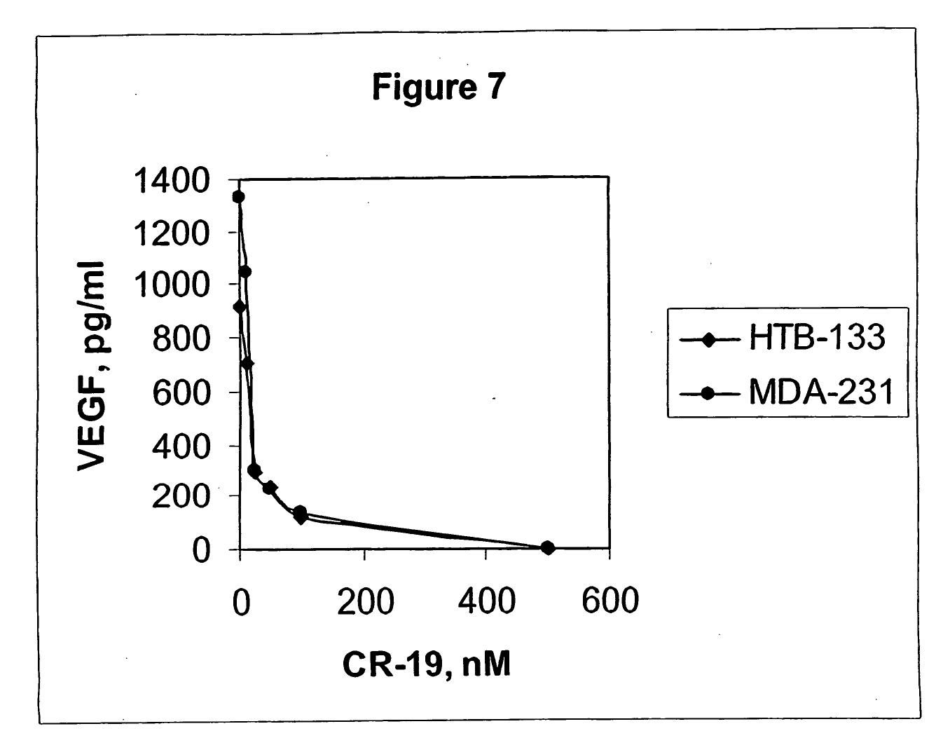

Results: Similar to CR-4, the compounds inhibited VEGF secretion in a dose response manner (FIGS. 6 and 7) and the IC50 values were approximately 30 nM, similar to CR4.

1. A method of inhibiting secretion of vascular endothelial growth factor in an animal in need of such inhibition, comprising administering to the animal an effective amount of a compound of Formula I, or a salt, solvate, prodrug, or hydrate thereof: 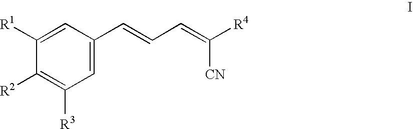
wherein
R¹ and R²are each independently selected from H, OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, O—Si(C₁₋₆alkyl)(C₁₋₆alkyl)(C₁₋₆alkyl), NO₂, CF₃, OCF₃ and halo;
R³ is selected from H, OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, O—Si(C₁₋₆alkyl)(C₁₋₆alkyl)(C₁₋₆alkyl), NO₂, halo and CH₂—S—(CH₂)ₙAr;
R⁴is selected from C(X)R⁵, SO₃Ar, NH₂, NH—C₁₋₆alkyl, N(C₁₋₆alkyl)(C₁₋₆alkyl), P(O)(OH)₂, P(O)(OC₁₋₆alkyl)₂, and C(NH₂)═C(CN)₂;
X is selected from O, S, NH and N—C₁₋₆alkyl;
R⁵ is selected from NH₂, OH, NH(CH₂)ₚAr, NH(CH₂)ₚOH, (CH₂)ₚOC₁₋₆alkyl, C₁₋₆alkyl, C₁₋₆alkoxy, NHNH₂, NHC(O)NH₂, NHC(O)C₁₋₆alkoxy, N-morpholino and N-pyrrolidino; and
Ar is an aromatic or heteroaromatic group, unsubstituted or substituted with 1-4 substituents, independently selected from OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, NO₂, CF₃, OCF₃ and halo;
n is 0 to 4; and
p is 1-4.

2. A method of inhibiting secretion of vascular endothelial growth factor in an animal in need of such inhibition, comprising administering to the animal an effective amount of a compound of Formula IV, or a salt, solvate, prodrug, or hydrate thereof: 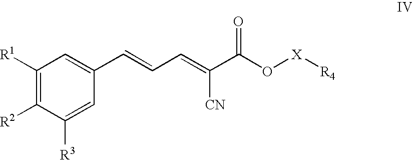
wherein
R¹, R² and R³ are each independently selected from H, OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, NO₂, CF₃, OCF₃ and halo;
R⁴ is unsubstituted Ar, or Ar substituted with 1-4 substituents, independently selected from C₁₋₆alkyl, C₁₋₆alkoxy and halo;
X is selected from (CH₂CH₂O)ₙ and (CH₂)ₙ; and
n is 1-4.

3. A method of inhibiting secretion of vascular endothelial growth factor in an animal in need of such inhibition, comprising administering to the animal an effective amount of a compound of Formula VI, and/or a salt, solvate, prodrug, or hydrate thereof: 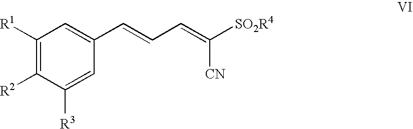
wherein
R¹, R² and R³ are each independently selected from H, OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, NO₂, CF₃, OCF₃ and halo; and
R⁴is selected from C₁₋₆alkyl, phenyl and pyridyl, wherein phenyl and pyridyl are unsubstituted or substituted with 1-4 substituents, independently selected from C₁₋₆alkyl, C₁₋₆alkoxy and halo.

4. A method of inhibiting an effect of vascular endothelial growth factor in an animal in need of such inhibition, comprising administering to the animal an effective amount of a compound of Formula I, or a salt, solvate, prodrug, or hydrate thereof: 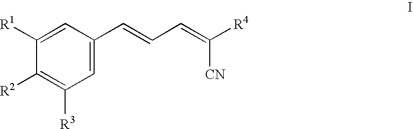
wherein
R¹ and R² are each independently selected from H, OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, O—Si(C₁₋₆alkyl)(C₁₋₆alkyl)(C₁₋₆alkyl), NO₂, CF₃, OCF₃ and halo;
R³is selected from H, OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, O—Si(C₁₋₆alkyl)(C₁₋₆alkyl)(C₁₋₆alkyl), NO₂, halo and CH₂—S—(CH₂)ₙAr;
R⁴is selected from C(X)R⁵, SO₃Ar, NH₂, NH—C₁₋₆alkyl, N(C₁₋₆alkyl)(C₁₋₆alkyl), P(O)(OH)₂, P(O)(OC₁₋₆alkyl)₂, and C(NH₂)═C(CN)₂;
X is selected from O, S, NH and N—C₁₋₆alkyl;
R⁵ is selected from NH₂, OH, NH(CH₂)ₚAr, NH(CH₂)ₚOH, (CH₂)ₚOC₁₋₆alkyl, C₁₋₆alkyl, C₁₋₆alkoxy, NHNH₂, NHC(O)NH₂, NHC(O)C₁₋₆alkoxy, N-morpholino and N-pyrrolidino; and
Ar is an aromatic or heteroaromatic group, unsubstituted or substituted with 1-4 substituents, independently selected from OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, NO₂, CF₃, OCF₃ and halo;
n is 0 to 4; and
p is 1-4.

5. A method of inhibiting an effect of vascular endothelial growth factor in an animal in need of such inhibition, comprising administering to the animal an effective amount of a compound of Formula IV, or a salt, solvate, prodrug, or hydrate thereof: 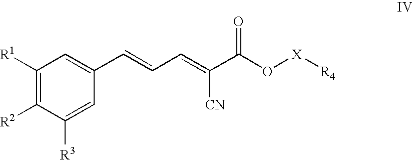
wherein
R¹, R² and R³ are each independently selected from H, OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, NO₂, CF₃, OCF₃ and halo;
R⁴ is unsubstituted Ar, or Ar substituted with 1-4 substituents, independently selected from C₁₋₆alkyl, C₁₋₆alkoxy and halo;
X is selected from (CH₂CH₂O)ₙ and (CH₂)ₙ, and
n is 1-4.

6. A method of inhibiting an effect of vascular endothelial growth factor in an animal in need of such inhibition, comprising administering to the animal an effective amount of a compound of Formula VI, and/or a salt, solvate, prodrug, or hydrate thereof: 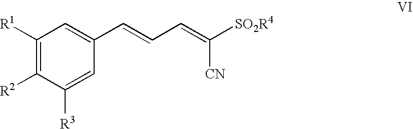
wherein
R¹, R² and R³ are each independently selected from H, OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, NO₂, CF₃, OCF₃ and halo; and
R⁴ is selected from C₁₋₆alkyl, phenyl and pyridyl, wherein phenyl and pyridyl are unsubstituted or substituted with 1-4 substituents, independently selected from C₁₋₆alkyl, C₁₋₆alkoxy and halo.

7. The method of claim 4, wherein the effect of vascular endothelial growth factor is angiogenesis, vasculogenesis, arteriogenesis, vascular permeability or inflammation.

8. A method of treating a disorder caused or contributed to by vascular endothelial growth factor in an animal in need of such treatment, comprising administering to the animal an effective amount of a compound of Formula I, or a salt, solvate, prodrug, or hydrate thereof: 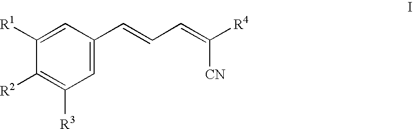
wherein
R¹ and R² are each independently selected from H, OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, O—Si(C₁₋₆alkyl)(C₁₋₆alkyl)(C₁₋₆alkyl), NO₂, CF₃, OCF₃ and halo;
R³ is selected from H, OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, O—Si(C₁₋₆alkyl)(C₁₋₆alkyl)(C₁₋₆alkyl), NO₂, halo and CH₂—S—(CH₂)ₙAr;
R⁴is selected from C(X)R⁵, SO₃Ar, NH₂, NH—C₁₋₆alkyl, N(C₁₋₆alkyl)(C₁₋₆alkyl), P(O)(OH)₂, P(O)(OC₁₋₆alkyl)₂, and C(NH₂)═C(CN)₂;
X is selected from O, S, NH and N—C₁₋₆alkyl;
R⁵ is selected from NH₂, OH, NH(CH₂)ₚAr, NH(CH₂)ₚOH, (CH₂)ₚOC₁₋₆alkyl, C₁₋₆alkyl, C₁₋₆alkoxy, NHNH₂, NHC(O)NH₂, NHC(O)C₁₋₆alkoxy, N-morpholino and N-pyrrolidino; and
Ar is an aromatic or heteroaromatic group, unsubstituted or substituted with 1-4 substituents, independently selected from OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, NO₂, CF₃, OCF₃ and halo;
n is 0 to 4; and
p is 1-4.

9. A method of treating a disorder caused or contributed to by vascular endothelial growth factor in an animal in need of such treatment, comprising administering to the animal an effective amount of a compound of Formula IV, or a salt, solvate, prodrug, or hydrate thereof: 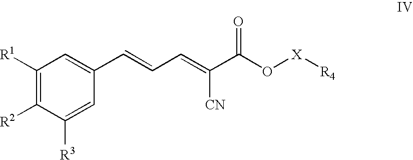
wherein
R¹, R² and R³ are each independently selected from H, OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, NO₂, CF₃, OCF₃ and halo;
R⁴ is unsubstituted Ar, or Ar substituted with 1-4 substituents, independently selected from C₁₋₆alkyl, C₁₋₆alkoxy and halo;
X is selected from (CH₂CH₂O)ₙ and (CH₂)ₙ, and
n is 1-4.

10. A method of treating a disorder caused or contributed to by vascular endothelial growth factor in an animal in need of such treatment, comprising administering to the animal an effective amount of a compound of Formula VI, a salt, solvate, prodrug, or hydrate thereof: 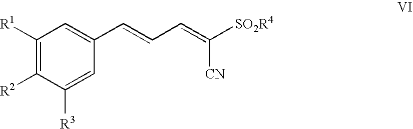
wherein
R¹, R² and R³ are each independently selected from H; OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, NO₂, CF₃, OCF₃ and halo; and
R⁴is selected from C₁₋₆alkyl, phenyl and pyridyl, wherein phenyl and pyridyl are unsubstituted or substituted with 1-4 substituents, independently selected from C₁₋₆alkyl, C₁₋₆alkoxy and halo.

11. (canceled)

12. The method of claim 8, wherein expression or levels of vascular endothelial growth factor are upregulated in the disorder.

13. The method of claim 8, wherein the disorder is cancer, rheumatoid arthritis, retinopathy, atherosclerosis, diabetes, corneal conjunctival vascularization, hemangioma, Karposi's sarcoma, endometriosis, psoriasis, hemotological malignancy, lymphoproliferative disorder, myeloproliferative disorder, renal vein occlusion, retinopathy of prematurity, age-related macular degeneration, or bullous disease.

14. The method of claim 13, wherein the disorder is cancer, and the cancer is a solid tumour cancer.

15. The method of claim 14, wherein the solid tumour cancer is breast cancer, pancreatic cancer, colon cancer or brain cancer.

16. The method of claim 14, wherein growth of a tumour is inhibited.

17-46. (canceled)

47. A medical device comprising:
(a) a substrate having a surface; and
(b) a coating disposed on the surface, said coating comprising a polymer matrix including a compound of Formula I, or a salt, solvate, prodrug, or hydrate thereof: 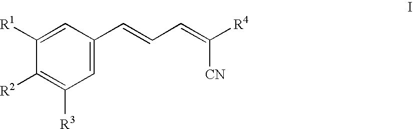
wherein
R¹ and R² are each independently selected from H, OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, O—Si(C₁₋₆alkyl)(C₁₋₆alkyl)(C₁₋₆alkyl), NO₂, CF₃, OCF₃ and halo;
R³ is selected from H, OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, O—Si(C₁₋₆alkyl)(C₁₋₆alkyl)(C₁₋₆alkyl), NO₂, halo and CH₂—S—(CH₂)ₙAr;
R⁴is selected from C(X)R⁵, SO₃Ar, NH₂, NH—C₁₋₆alkyl, N(C₁₋₆alkyl)(C₁₋₆alkyl), P(O)(OH)₂, P(O)(OC₁₋₆alkyl)₂, and C(NH₂)═C(CN)₂;
X is selected from O, S, NH and N—C₁₋₆alkyl;
R⁵ is selected from NH₂, OH, NH(CH₂)ₚAr, NH(CH₂)ₚOH, (CH₂)ₚOC₁₋₆alkyl, C₁₋₆alkyl, C₁₋₆alkoxy, NHNH₂, NHC(O)NH₂, NHC(O)C₁₋₆alkoxy, N-morpholino and N-pyrrolidino; and
Ar is an aromatic or heteroaromatic group, unsubstituted or substituted with 1-4 substituents, independently selected from OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, NO₂, CF₃, OCF₃ and halo;
n is 0 to 4; and
p is 1-4.

48-52. (canceled)

53. The method of claim 1, wherein said animal has, or is at risk for developing, arteriovenous malformations (AVM), meningioma, vascular restenosis, angiofibroma, dermatitis, endometriosis, hemophilic joints, hypertrophic scars, inflammatory disease, pyogenic granuloma, scleroderma, synovitis, trachoma or vascular adhesions.

54. The method of claim 1, wherein said animal has, or is at risk for developing, abnormal proliferation of fibrovascular tissue, acne rosacea, acquired immune deficiency syndrome, artery occlusion, atopic keratitis, bacterial ulcers, Bechets disease, blood borne tumors, carotid obstructive disease, chemical burns, choroidal neovascularization, chronic inflammation, chronic retinal detachment, chronic uveitis, chronic vitritis, contact lens overwear, corneal graft rejection, corneal neovascularization, corneal graft neovascularization, Crohn's disease, Eales disease, epidemic keratoconjunctivitis, fungal ulcers, Herpes simplex infections, Herpes zoster infections, hyperviscosity syndromes, Kaposi's sarcoma, leukemia, lipid degeneration, Lyme's disease, marginal keratolysis, Mooren ulcer, Mycobacteria infections other than leprosy, myopia, ocular neovascular disease, optic pits, Osler-Weber syndrome (Osler-Weber-Rendu), osteoarthritis, Pagets disease, pars planitis, pemphigoid, phylectenulosis, polyarteritis, post-laser complications, protozoan infections, pseudoxanthoma elasticum, pterygium keratitis sicca, radial keratotomy, retinal neovascularization, retinopathy of prematurity, retrolental fibroplasias, sarcoid, scleritis, sickle cell anemia, Sogrens syndrome, solid tumors, Stargarts disease, Steven's Johnson disease, superior limbic keratitis, syphilis, systemic lupus, Terrien's marginal degeneration, toxoplasmosis, trauma, tumors of Ewing sarcoma, tumors of neuroblastoma, tumors of osteosarcoma, tumors of retinoblastoma, tumors of rhabdomyosarcoma, ulcerative colitis, vein occlusion, Vitamin A deficiency or Wegeners sarcoidosis.

55. The method of claim 1, wherein said animal has, or is at risk for developing, diabetes; parasitic disease; abnormal wound healing; hypertrophy following surgery, burns, injury or trauma; inhibition of hair growth; inhibition of ovulation and corpus luteum formation; inhibition of implantation or inhibition of embryo development in the uterus.

56. The method of claim 1, wherein said animal has, or is at risk for developing, graft rejection, lung inflammation, nephrotic syndrome, preeclampsia, edema associated with brain tumors, ascites associated with malignancies, Meigs' syndrome, pericardial effusion, pericarditis or pleural effusion.

57. The method of claim 1, wherein said animal has, or is at risk for developing, a vascularized solid tumor, a metastatic tumor or metastases from a primary tumor.

58. The method of claim 57, further comprising administering to said animal a therapeutically effective amount of at least a second anti-cancer agent.

59. The method of claim 58, wherein said at least a second anti-cancer agent is a chemotherapeutic agent, radiotherapeutic agent, anti-angiogenic agent, apoptosis-inducing agent or anti-tubulin drug or a tumor-targeted chemotherapeutic agent, radiotherapeutic agent, anti-angiogenic agent, apoptosis-inducing agent or anti-tubulin drug.

60. The method of claim 59, wherein said at least a second anti-cancer agent is an anti tubulin drug selected from the group consisting of colchicine, taxol, vinblastine, vincristine, vindescine and a combretastatin or a tumor-targeted anti-tubulin drug selected from the group consisting of colchicine, taxol, vinblastine, vincristine, vindescine and a combretastatin.

61. The method of claim 1, for treating endometriosis.

62. The method of claim 61, wherein the compound is administered by an intra-uterine or intra-peritoneal route.

63. The method of claim 1, for treating an ocular neovascular disease in a patient.

64. The method of claim 63, wherein said neovascular disease is selected from the group consisting of ischemic retinopathy, intraocular neovascularization, age-related macular degeneration, corneal neovascularization, retinal neovascularization, choroidal neovascularization, diabetic macular edema, diabetic retina ischemia, diabetic retinal edema, and proliferative diabetic retinopathy.

65. (canceled)

66. The method of claim 1, for treating atherosclerosis in a patient.

67. The method of claim 1, for treating an inflammatory disease in a patient.

68. The method of claim 67, wherein the inflammatory disease is arthritis.

69. A method of interfering with angiogenesis, neovascularization or lymphangiogenesis in a mammal having a condition characterized by angiogenesis, neovascularization or lymphangiogenesis, comprising administering to said mammal an effective amount of a compound represented in Formula I, or a salt, solvate, prodrug, or hydrate thereof: 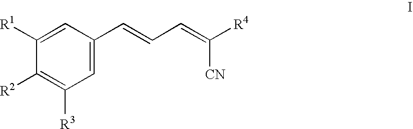
wherein
R¹ and R² are each independently selected from H, OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, O—Si(C₁₋₆alkyl)(C₁₋₆alkyl)(C₁₋₆alkyl), NO₂, CF₃, OCF₃ and halo;
R³ is selected from H, OH, C₁₋₆alkyl, C₁₋₆alkoxy, NH₂, NH—C₁₋₆alkyl, N(C₁₋₆alkyl)(C₁₋₆alkyl), SH, S—C₁₋₆alkyl, O—Si(C₁₋₆alkyl)(C₁₋₆alkyl)(C₁₋₆alkyl), NO₂, halo and CH₂—S—(CH₂)ₙAr;
R⁴is selected from C(X)R⁵, SO₃Ar, NH₂, NH—C₁₋₆alkyl, N(C₁₋₆alkyl)(C₁₋₆alkyl), P(O)(OH)₂, P(O)(OC₁₋₆alkyl)₂, and C(NH₂)═C(CN)₂;
X is selected from O, S, NH and N—C₁₋₆alkyl;
R⁵ is selected from NH₂, OH, NH(CH₂)ₚAr, NH(CH₂)ₚOH, (CH₂)ₚOC₁₋₆alkyl, C₁₋₆alkyl, C₁₋₆alkoxy, NHNH₂, NHC(O)NH₂, NHC(O)C₁₋₆alkoxy, N-morpholino and N-pyrrolidino; and
Ar is an aromatic or heteroaromatic group, unsubstituted or substituted with 1-4 substituents, independently selected from OH, C₁₋₆alkyl, C₁₋₆alkoxy, NH₂, NH—C₁₋₆alkyl, N(C₁₋₆alkyl)(C₁₋₆alkyl), SH, S—C₁₋₆-alkyl, NO₂, CF₃, OCF₃ and halo;
n is 0 to 4;
and p is 1-4.
